(19) United States
(12) Patent Application Publication
(10) Pub. No.: US 2006/0024347 A1
(43) Pub. Date: Feb. 2, 2006

(54) Bioactive peptide coatings

(75) Inventors: Paul O. Zamora, Gaithersburg, MD (US); Sarah Campion, Alexandria, VA (US)

Correspondence Address: PEACOCK MYERS, P.C., 201 THIRD STREET, N.W., SUITE 1340, ALBUQUERQUE, NM, 87102, US

(73) Assignee: BioSurface Engineering Technologies, Inc., College Park, MD (US)

(21) Appl. No.: 11/167,636

(22) Filed: Jun. 27, 2005

Related U.S. Application Data
(63) Continuation-in-part of application No. 11/055,428, filed on Feb. 10, 2005.
(60) Provisional application No. 60/543,616, filed on Feb. 10, 2004.
(60) Provisional application No. 60/583,566, filed on Jun. 28, 2004.
(60) Provisional application No. 60/655,570, filed on Feb. 22, 2005.
(60) Provisional application No. 60/656,174, filed on Feb. 25, 2005.

(51) Int. Cl. A61K 38/24; B05D 3/00; A61F 2/00

(52) U.S. Cl. 424/423; 530/399; 427/2.24

(57) ABSTRACT

The invention provides a coating and a method for coating medical devices with synthetic heparin-binding growth factor analogs, the coating and method including at least one heparin-binding growth factor analog with a region of amino acid residues binding a heparin-binding growth factor receptor, a hydrophobic linker region and a heparin-binding region, and further including heparin or an analog thereof. Also provided are medical devices, including aneurysm coils, coated with synthetic heparin-binding growth factor analogs, the coating further including heparin or an analog thereof.

### CROSS-REFERENCE TO RELATED APPLICATIONS

This application is a continuation-in-part application of U.S. patent application Ser. No. 11/055,428, entitled “Heterodimeric Chain Synthetic Heparin-Binding Growth Factor Analogs”, filed on Feb. 10, 2005, which in turn claims priority to and the benefit of the filing of U.S. Provisional Patent Application Ser. No. 60/543,616, entitled “Heterodimeric Chain Synthetic Heparin-Binding Growth Factor Analogs”, filed on Feb. 10, 2004, and the specification and claims thereof of each are incorporated herein by reference.

This application claims priority to and the benefit of the filing of U.S. Provisional Patent Application Ser. No. 60/583,566, entitled “Bioactive Peptide Coatings”, filed on Jun. 28, 2004; U.S. Provisional Patent Application Ser. No. 60/655,570, entitled “Single Branch Heparin-Binding Growth Factor Analogs”, filed on Feb. 24, 2005; and U.S. Provisional Patent Application Ser. No. 60/656,174, entitled “TGF Growth Factor Analogs”, filed on Feb. 25, 2005; and the specification and claims thereof of each are incorporated herein by reference.

### BACKGROUND OF THE INVENTION

1. Field of the Invention (Technical Field)

The present invention relates to methods for coating and coatings for medical devices comprising synthetic peptides and analogs of heparin-binding growth factors, particularly analogs further having a heparin-binding region and hydrophobic linker, and further comprising heparin or a heparin analog.

2. Description of Related Art

Note that the following discussion refers to a number of publications by author(s) and year of publication, and that due to recent publication dates certain publications are not to be considered as prior art vis-a-vis the present invention. Discussion of such publications herein is given for more complete background and is not to be construed as an admission that such publications are prior art for patentability determination purposes.

The heparin-binding growth factors (HBGFs) constitute a large class of growth factors that includes the 23 fibroblast growth factors identified to date (FGFs 1-23), HBBM (heparin-binding brain mitogen), HB-GAF (heparin-binding growth associated factor), HB-EGF (heparin-binding EGF-like factor) HB-GAM (heparin-binding growth associated molecule), TGF-α (transforming growth factor-α), TGF-βs (transforming growth factor-βs), PDGF (platelet-derived growth factor), EGF (epidermal growth factor), VEGF (vascular endothelial growth factor), IGF-1 (insulin-like growth factor-1), IGF-2 (insulin-like growth factor-2), HGF (hepatocyte growth factor), IL-1 (interleukin-1), IL-2 (interleukin-2), IFN-α (interferon-α), IFN-γ (interferon-γ), TNF-α (tumor necrosis factor-α), SDGF (Schwannoma-derived growth factor) and the many other growth factors, cytokines, lymphokines and chemokines that have an affinity for heparin.

Peptides from natural HBGFs that bind heparin-binding growth factor receptors have been identified. See for example Ray et al., Proc. Natl. Acad. Sci. USA 94:7047-7052 (1997). These authors demonstrated that two amino acid sequences from FGF-2 are sufficient to block the mitogenic activity of FGF-2 on neural progenitor cells. The first peptide is a ten amino acid sequence, from amino acids 65-74, the second peptide extends from amino acids 115-129.

In an alternative approach, an artificial peptide that binds a heparin-binding growth factor receptor was identified by a phage display method. Ballinger et al., Nature BioTechnology 17:1199-1204 (1999) used this technique to isolate a 28 amino acid peptide called C19, binds FGF-2 receptors, but by itself fails to stimulate biological activity. The peptide has no amino acid sequence identity with any known FGF.

HBGFs useful in prevention or therapy of a wide range of diseases and disorders may be purified from natural sources or produced by recombinant DNA methods, however, such preparations are expensive and generally difficult to prepare.

Some efforts have been made to generate heparin-binding growth factor analogs. For example, natural PDGF occurs as an A chain and a B chain arranged in head-to-head (AA or BB) homodimers, or (AB or BA) heterodimers. Thus, U.S. Pat. No. 6,350,731 to Jehanli et al. discloses PDGF analogs in which two synthetic PDGF receptor-binding domains are covalently linked through a polyglycine or an N-(4-carboxy-cyclohexylmethyl)-maleimide (SMCC) chain to mimic the natural active polypeptide dimer.

U.S. Pat. No. 6,235,716 to Ben-Sasson discloses analogs of angiogenic factors. The analogs are branched multivalent ligands that include two or more angiogenic homology regions connected by a multilinker backbone.

U.S. Pat. No. 5,770,704 (the '704 patent) to Godowski discloses conjugates for activating receptor tyrosine kinases, cytokine receptors and members of the nerve growth factor receptor superfamily. The conjugates include at least two ligands capable of binding to the cognate receptor, so that the binding of the respective ligands induces oligomerization of these receptors. The ligands disclosed in the '704 patent are linked by covalent attachment to various nonproteinaceous polymers, particularly hydrophilic polymers, such as polyvinylalcohol and polyvinylpyrrolidone, and the polyvinylalkene ethers, including polyethylene glycol and polypropylene glycol. The ligands include hepatocyte growth factor (HGF) peptide variants that each bind HGF receptor, thereby causing receptor dimerization and activation of the biological activity of the HGF receptor dimer.

U.S. Pat. No. 6,284,503 (the '503 patent) to Caldwell et al. discloses a composition and method for regulating the adhesion of cells and biomolecules to hydrophobic surfaces and hydrophobic coated surfaces for cell adhesion, cell growth, cell sorting and biological assays. The composition is a biomolecule conjugated to a reactive end group activated polymer. The end group activated polymer includes a block copolymer surfactant backbone and an activation or reactive group. The block copolymer may be any surfactant having a hydrophobic region capable of adsorbing onto a hydrophobic surface, and a hydrophilic region which extends away from the surface when the hydrophobic region is adsorbed onto the hydrophobic surface. The '503 patent discloses that the biomolecules that may be conjugated to the end group activated polymer include natural or recombinant growth factors, such as PDGF, EGF, TGFα, TGFβ, NGF, IGF-I, IGF-II, GH and GHRF, as well as multi-CSF(II-3), GM-CSF, G-CSF, and M-CSF.

Other workers have described compositions that include homologs and analogs of fibroblast growth factors (FGFs). See for example U.S. Pat. No. 5,679,673 to Lappi and Baird; U.S. Pat. No. 5,989,866 to Deisher et al. and U.S. Pat. No. 6,294,359 to Fiddes et al. These disclosures relate to FGF homologs or analogs that are either conjugated to a toxic moiety and are targeted to the FGF receptor-bearing cells; or are homologs or analogs that modulate the biological pathways through the signal transduced by the FGF receptor upon binding by the FGF homolog or analog.

A series of patent applications to Kochendoerfer et al. disclose polymer-modified proteins, including synthetic chemokines and erythropoiesis stimulating proteins. See, for example, International Publications WO 02/04105, WO 02/19963 and WO 02/20033. These include chemically ligated peptide segments of a polypeptide chain of a synthetic erythropoiesis protein, such that a polypeptide chain results, with a water soluble polymer attached at one or more glycosylation sites on the protein. These applications also disclose synthetic chemokines, which are also polymer modified, and are asserted to be antagonists. However, heparin-binding domains are not disclosed. Other erythropoietin mimetics are known, such as those disclosed in U.S. Pat. Nos. 5,773,569 and 5,830,851 to Wrighton et al.

International Publication WO 00/18921 to Ballinger and Kavanaugh discloses a composition consisting of fusion proteins having FGF receptor affinity linked to an “oligomerization domain”, either directly or through a linking group. The oligomerization domain ranges in length from about 20 to 300 residues, and includes constructs such as transcription factors, Fc portions of IgG, leucine zippers and the like. The oligomerization domains disclosed are homodimeric domains, wherein a single FGF receptor affinity fusion protein is linked to a single domain, such as a leucine zipper, which in turn is linked to a similar molecule by means of cysteine residues at both the amino and carboxy termini of the leucine zippers, such that two parallel leucine zippers, each with a single FGF receptor affinity fusion protein, are cross-linked by means of disulfide bonds. It is also disclosed that fusion proteins may include a heparin binding domain, such as the use of jun as a multimerization domain, which is asserted to be a heparin binding domain. Thus the compositions disclosed by Ballinger and Kavanaugh are all composed of a single receptor-binding sequence covalently attached to an oligomerization domain, whereby two or more similar oligomerization domains, each with a single receptor-binding sequence, are conjoined by means of either an association provided by the oligomerization domain, or alternatively, are chemically cross-linked to provide for the covalent bonding of the individual components.

U.S. Patent Application Publication Nos. 2004/0087505 A1 and 2004/0038348 A1, and International Published Patent Application Serial No. WO 2004/018499 A2, each incorporated herein by reference, disclose synthetic heparin-binding growth factor analogs that not only include a receptor binding domain but also include both a hydrophobic linker, providing for the linking of two receptor-binding domains to a dipeptide sequence, and a single non-signaling peptide containing a heparin-binding domain. One disclosed method of coating is where a heparin-containing compound, such as benzyl-bis(dimethylsilylmethyl)oxycarbamoyl-heparin, is first bonded to the surface of the medical device, and the heparin-binding domain of the synthetic heparin-binding growth factor analog then binds the heparin-containing compound. This method generally provides for a slow and steady release of the bound composition after placing the medical device in a patient. However, there is still a need for an effective coating method that facilitates the release of bound compositions within a desired time after placing the medical device in a patient. In particular, there is a need for an effective coating method that facilitates the quick release of a synthetic heparin-binding growth factor analog useful for coating medical devices and soluble biologics.

### SUMMARY OF THE INVENTION

In one embodiment, the invention provides a method of coating a medical device on a surface thereof, the method including the steps of: contacting a medical device with a first solution including a synthetic heparin-binding growth factor analog comprising at least one sequence of amino acid residues binding a heparin-binding growth factor receptor (HBGFR), a hydrophobic linker region and a heparin-binding region; and contacting the medical device with a second solution including a heparin-containing compound. The method may further include the step of contacting the medical device with a third solution comprising a heparin-binding growth factor analog including at least one sequence of amino acid residues binding a HBGFR, a hydrophobic linker region and a heparin-binding region. In the method, heparin-binding growth factor analog of the first solution be the same as the heparin-binding growth factor analog of the third solution, or may be different. In one preferred embodiment of the method, the steps are ordered such that the medical device is first contacted with a first solution, second contacted with a second solution, and third contacted with a third solution. In the method, the medical device can be a suture, graft material, wound covering, nerve guide, bone wax, aneurysm coil, embolization particle, microbead, stent, dental implant, bone prosthesis, tissue scaffold or controlled release drug delivery device. The surface of the medical device can be stainless steel, titanium, platinum, tungsten, ceramics, polyurethane, polytetrafluoroethylene, extended polytetrafluoroethylene, polycarbonate, polyester, polypropylene, polyethylene, polystyrene, polyvinyl chloride, polyamide, polyacrylate, polyurethane, polyvinyl alcohol, polycaprolactone, polyactide, polyglycolide, polysiloxane, natural rubber, artificial rubber, a block polymer, or a copolymer of block polymers. In one embodiment of the method, the heparin-containing compound includes a silyl-heparin compound. In one embodiment of the method, the at least one sequence of amino acid residues binding a HBGFR is a sequence selected from the group consisting of SEQ ID NO:7 to SEQ ID NO:70. In another embodiment of the method, the heparin-binding region is a sequence of amino acid residues selected from the group consisting of SEQ ID NO:1 to SEQ ID NO:6. In yet another embodiment of the invention, the synthetic heparin-binding growth factor analog including at least one sequence of amino acid residues binding a HBGFR, a hydrophobic linker region and a heparin-binding region is a construct of any of formulas I to XI.

In another embodiment, the invention provides a method of coating a medical device on a surface thereof, the method including the step of: contacting a medical device with a first solution including a heparin-containing compound and a synthetic heparin-binding growth factor analog including at least one sequence of amino acid residues binding a heparin-binding growth factor receptor (HBGFR), a hydrophobic linker region and a heparin-binding region. This method may further include the step of contacting the medical device with a second solution including a heparin-binding growth factor analog including at least one sequence of amino acid residues binding a heparin-binding growth factor receptor (HBGFR), a hydrophobic linker region and a heparin-binding region.

Another embodiment of the present invention provides a coated medical device including a medical device member and a coating including the product of application of a synthetic heparin-binding growth factor analog including at least one sequence of amino acid residues binding a HBGFR, a hydrophobic linker region and a heparin-binding region and sequential application of a heparin-containing compound. The coated medical device may further include a coating that is product of a sequential application of a synthetic heparin-binding growth factor analog including at least one sequence of amino acid residues binding a HBGFR, a hydrophobic linker region and a heparin-binding region. In a coated medical device the at least one sequence of amino acid residues binding a HBGFR may be a sequence selected from the group consisting of SEQ ID NO:7 to SEQ ID NO:70. A heparin-binding region may be a sequence of amino acid residues selected from the group consisting of SEQ ID NO:1 to SEQ ID NO:6. A hydrophobic linker region may include amino hexanoic acid. A heparin-containing compound may include a silyl-heparin compound, including but not limited to benzyl-bis(dimethylsilylmethyl)oxycarbamoyl-heparin. The coating of a coated medical device may further be characterized in that it binds a cell surface receptor, supports cell attachment, or is vaso-occlusive. A medical device may be a suture, graft material, wound covering, nerve guide, bone wax, aneurysm coil, embolization particle, microbead, stent, dental implant, bone prosthesis, tissue scaffold or controlled release drug delivery device.

Another embodiment of the present invention provides a coating for a medical device, the coating including a composition formed in situ from the sequential application to the medical device of a first solution including a synthetic heparin-binding growth factor analog including at least one sequence of amino acid residues binding a HBGFR, a hydrophobic linker region and a heparin-binding region and a second solution including a heparin-containing compound. The coating may further include a composition formed in situ from the sequential application to the medical device of a third solution including a heparin-binding growth factor analog including at least one sequence of amino acid residues binding a HBGFR, a hydrophobic linker region and a heparin-binding region.

Another embodiment of the present invention provides a coating for a medical device, the coating comprising a HBGF analog including at least one sequence binding a heparin-binding growth factor receptor (HBGFR), a hydrophobic linker region, and a heparin-binding region. The coating further includes heparin, wherein the heparin is applied after the HBFG analog is applied. The coating can further optionally include a second application of a HBGF analog, which may be the same as the first HBGF analog or different.

The invention further includes a medical device with a coating as described.

The invention further includes a method of coating a medical device, including the step of contacting a medical device with a solution including a HBGF analog. It is hypothesized, without wishing to be bound by theory, that the HBGF analog is non-covalently bound to the medical device by hydrophobic interaction with the hydrophobic linker region of the HBGF analog providing such hydrophobic interaction. In the method, heparin is subsequently contacted with the HBGF analog-coated medical device, such as by contacting with a solution including heparin or an analog thereof. It is hypothesized, again without wishing to be bound by theory, that the heparin forms a complex with the heparin-binding region of the HBGF analog. The method may further include the step of contacting the HBGF analog and heparin coated medical device with a solution including a second HBGF analog. In a preferred embodiment, the second HBGF analog is the same as the initial coating HBGF analog. It is hypothesized, again without wishing to be bound by theory, that the second HBGF analog is bound to the coating complex of the initial HBGF analog and heparin by means of a complex between available binding regions on the heparin and the heparin-binding regions of the second HBGF analog.

According to one embodiment of the present invention, the following steps are provided:

a) contacting a medical device with a solution including a HBGF analog;

b) contacting the HBGF analog-coated medical device with a solution comprising heparin or analog thereof; and, c) contacting the product of step b) with a solution including a second HBGF analog, optionally wherein the second HBGF is the same as the HBGF analog of step a).

In yet another embodiment, a HBGF analog is allowed to react with a solution including heparin or an analog thereof, and a medical device is subsequently contacted with such solution.

In yet another embodiment, the HBGF analog is the construct hereafter named F2A3 and the medical device is an aneurysm coil.

According to another embodiment of the present invention a method for delivering an active peptide to a mammal, particularly a human, is provided. The method includes providing a medical device coated on the surface thereof via non-covalent bonds with a HBGF analog and placing the medical device onto a surface of, or implanting the medical device into, the mammal. The method also includes providing a medical device coated on the surface thereof via non-covalent bonds with a HBGF analog complexed to heparin. The method further includes providing a medical device coated on the surface thereof via non-covalent bonds with a HBGF analog, with sequential complexation to heparin and a second HBGF analog, preferably wherein the second HBGF analog is the same as the initial HBGF analog. The method also includes providing a medical device coated on the surface thereof via non-covalent bonds with a HBGF analog and complexation with a pre-formed heparin-peptide complex. Preferably the peptide in the pre-formed heparin-peptide complex is a HBGF analog.

It is an object of the present invention to provide a method for coating medical devices with HBGF analogs.

It is a further object to provide a method that does not involve chemical conjugation steps to coat a medical device, but rather relies upon hydrophobic interaction and formation of a heparin-binding complex.

It is another object to provide a method for coating medical devices with a HBGF analog and heparin complex wherein the coating involves simple “dip” steps wherein the medical device to be coated is sequentially placed is specified solutions for specified periods of time.

It is another object to provide a method which can provide for desired pharmacokinetic and biodistribution patterns of HBGF analogs released from coatings for medical devices.

It is yet another object of the present invention to provide a low cost, simple and widely applicable method for coating medical devices with HBGF analogs.

Other objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 depicts the sequence of synthetic peptide analog F2A3.

FIG. 2 depicts the sequence of synthetic peptide analog F2A4.

FIG. 3 is a plot of the elution of F2A3 from a heparin affinity column.

FIG. 4A is a graph depicting specific binding of F2A3 to FGFRs on HUVECs; FIG. 4B is a graph depicting specific binding of F2A3 and F2A4 to FGFRs on C3H10T1/2 fibroblasts.

FIG. 5 is a blot illustrating the equivalence of FGF-2 analogs F2A3 and F2A4 to native, recombinant FGF-2 in MAP kinase phosphorylation and activation.

FIG. 6 is a graph of stimulation of cell proliferation in fibroblast cultures, illustrating the mitogenic dose response of F2A3 and F2A4 versus FGF-2.

FIG. 7A is a plot illustrating that F2A3 and F2A4 mimic FGF-2 for cell attachment in vitro, showing attachment, after two hours, of CH310T1/2 murine fibroblasts to polystyrene coated with silyl-heparin alone or with silyl-heparin plus FGF-2 or F2A3. (*) indicates p less than 0.05; FIG. 7B is a micrograph of bovine aortic endothelial cells grown on polycaprolactone with (left panel) and without (right panel) a coating of F2A3.

FIG. 8A is a plot illustrating the comparison of capillaries/field utilizing coated polylactide sutures in rat muscle at 2 weeks, comparing no coating with sutures coated with silyl heparin plus F2A3; FIG. 8B are micrographs of coated polylactide sutures in rat muscle at 2 weeks, where panel A is no coating, panel B a silyl heparin coating, panel C F2A3 coating, and panel D, silyl heparin and F2A3 coating.

FIG. 9 is a plot of the elution of F2A3 from differently coated stainless steel wafers starting at 0, 1, 2, 3 or 4 days after coating. The wafers were coated with F2A3, F2A3 and heparin, or F2A3 and heparin followed by a second coat of F2A3. F2A3 was detected by ELISA at an absorbance of 450 nm.

### DETAILED DESCRIPTION OF THE INVENTION

Each synthetic HBGF analog utilized in the invention is an analog of a particular HBGF that binds to one or more of the receptors bound by the particular HBGF. The synthetic HBGF analog may be an analog of a hormone, a cytokine, a lymphokine, a chemokine or an interleukin.

In one aspect the synthetic HBGF analog of the present invention is a molecule comprising amino acid residues binding a heparin-binding growth factor receptor (HBGFR), a hydrophobic linker region and a heparin-binding region. HBGFs include any growth factor that binds selectively to heparin. For example, the HBGF can be any of the known FGFs (FGF-1 to FGF-23), HBBM (heparin-binding brain mitogen), HB-GAF (heparin-binding growth associated factor), HB-EGF (heparin-binding EGF-like factor) HB-GAM (heparin-binding growth associated molecule, also known as pleiotrophin, PTN, HARP), TGF-α60 (transforming growth factor-α), TGF-βs (transforming growth factor-βs), VEGF (vascular endothelial growth factor), EGF (epidermal growth factor), IGF-1 (insulin-like growth factor-1), IGF-2 (insulin-like growth factor-2), PDGF (platelet derived growth factor), RANTES, SDF-1, secreted frizzled-related protein-1 (SFRP-1), small inducible cytokine A3 (SCYA3), inducible cytokine subfamily A member 20 (SCYA20), inducible cytokine subfamily B member 14 (SCYB14), inducible cytokine subfamily D member 1 (SCYD1), stromal cell-derived factor-1 (SDF-1), thrombospondins 1, 2, 3 and 4 (THBS1-4), platelet factor 4 (PF4), lens epithelium-derived growth factor (LEDGF), midikine (MK), macrophage inflammatory protein (MIP-1), moesin (MSN), hepatocyte growth factor (HGF, also called SF), placental growth factor, IL-1 (interleukin-1), IL-2 (interleukin-2), IL-3 (interleukin-3), IL-6 (interleukin-6), IL-7 (interleukin-7), IL-10 (interleukin-10), IL-12 (interleukin-12), IFN-α (interferon-α), IFN-γ (interferon-γ), TNF-α (tumor necrosis factor-α), SDGF (Schwannoma-derived growth factor), nerve growth factor, neurite growth-promoting factor 2 (NEGF2), neurotrophin, BMP-2 (bone morphogenic protein 2), OP-1 (osteogenic protein 1, also called BMP-7), keratinocyte growth factor (KGF), interferon-γ inducible protein-20, RANTES, and HIV-tat-transactivating factor, amphiregulin (AREG), angio-associated migratory cell protein (AAMP), angiostatin, betacellulin (BTC), connective tissue growth factor (CTGF), cysteine-rich angiogenic inducer 61 (CYCR61), endostatin, fractalkine/neuroactin, or glial derived neurotrophic factor (GDNF), GRO2, hepatoma-derived growth factor (HDGF), granulocyte-macrophage colony stimulating factor (GMCSF), and the many growth factors, cytokines, interleukins and chemokines that have an affinity for heparin.

The amino acid sequences of many of these and other HBGFs are available from the National Library of Medicine Protein Database at the internet site www.ncbi.nlm.nih.gov/entrez. These HBGF amino acid sequences on the foregoing internet site are hereby incorporated by reference. The use of synthetic HBGF analogs incorporating the amino acid sequences of the receptor binding domains from these and other HBGFs is specifically contemplated in the present invention.

In particular embodiments of the present invention, the HBGF analog is a synthetic analog comprising amino acid residues binding a HBGFR, a hydrophobic linker region and a heparin-binding region. Thus in particular embodiments the synthetic HBGF analog of the present invention may consist of constructs of any of the following formulas.

In one embodiment, the coating and methods utilize a HBGF analog of formula I: 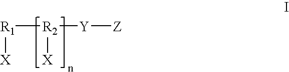
wherein each X is a peptide chain that (i) has a minimum of three amino acid residues, (ii) has a maximum of about fifty amino acid residues, and (iii) binds a heparin-binding growth factor receptor (HBGFR); R₁ is an amino acid residue, wherein X is covalently bonded through the N-terminus of R₁ or through a side chain of R₁; R₂ is a trifunctional alpha amino acid residue, wherein X is covalently bonded through a side chain of R₂; Y is a linker comprising a chain from 0 to about 50 atoms covalently bonded to R₁ and Z when n=0, or to R₂ and Z when n=1; Z is a non-signaling peptide chain that comprises a heparin binding domain, comprising an amino acid sequence that comprises (i) a minimum of one heparin binding motif, (ii) a maximum of about ten heparin binding motifs, and (iii) a maximum of about thirty amino acids; and, n is 0 or 1, wherein when n=1 the peptide chains X are identical.

In the HBGF analog of formula I, Y is a linker that (i) is hydrophobic, (ii) comprises a chain of a minimum of about 9 and a maximum of about 50 atoms, and (iii) is preferably not found in the natural ligand of the HBGFR which X binds. In one embodiment of formula I, R₁ is a trifunctional amino acid residue, wherein X is covalently bonded through a side chain of R₁.

In one embodiment of formula I, the HBGF analog of formula I is characterized in that it has an avidity for heparin such that the it binds heparin in 0.15 M NaCl, but is eluted by 1 M NaCl.

In another embodiment, the coating and methods utilize aHBGF analog of formula II: 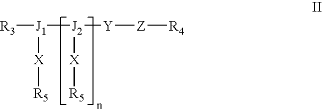
wherein R₃ and R₅ are each independently NH₂, an acyl group with a linear or branched C₁ to C₁₇ alkyl, aryl, heteroaryl, alkene, alkenyl or aralkyl chain including a N-terminus NH₂, NH₃⁺, or NH group or a corresponding acylated derivative, or is an amino acid, a dipeptide or a tripeptide, with an N-terminus NH₂, NH₃⁺, NH group or a corresponding acylated derivative; R₄ is —OH, NH₂, NH—R₆, or is an amino acid, a dipeptide or a tripeptide with a C-terminus —OH, NH₂, or NH—R₆; R₆ is an aliphatic C₁ to C₁₇ chain; each X is a peptide chain defined as above; J₁ and J₂ are each independently a trifunctional alpha amino acid residue, wherein each X is covalently bonded through a side chain of J₁ or J₂; Y is a linker defined as above covalently bonded to J₁ and Z when n=0, or to J₂ and Z when n=1; Z is a non-signaling peptide defined as above; and, n is 0 or 1, wherein when n=1 the synthetic peptide chains X are identical.

In the HGBF analog of formula II, Y is a linker that (i) hydrophobic, (ii) comprises a chain of a minimum of about 9 and a maximum of about 50 atoms, and (iii) is not preferably found in the natural ligand of the HBGFR which X binds.

In one embodiment, the HBGF analog of formula II is characterized in that it has an avidity for heparin such that it binds heparin in 0.15 M NaCl, but is eluted by 1 M NaCl.

The HBGF analog of formula II can further be characterized in that binding of it to the HBGFR initiates a signal by the HBGFR, or alternatively in that it blocks signaling by the HBGFR.

In one embodiment of the HBGF analog of formula II, J₁ and, if n=1, J₂, is a diamine amino acid residue. Such diamine amino acid residue may be a 2,3 diamino propionyl amino acid residue, a lysyl residue or an ornithinyl residue. In an alternative embodiment of the HBGF analog of formula II, the side chain of J₁ and, if n=1, J₂, includes a reactive carboxyl group.

In one embodiment of the HBGF analog of formula II, the covalent bond between X and J₁ or, if n=1, J₂, comprises an amide, disulfide, thioether, Schiff base, reduced Schiff base, imide, secondary amine, carbonyl, urea, hydrazone or oxime bond. In a preferred embodiment, the bond is an amide bond.

The HBGF analog of formula II thus includes a HBGF analog of formula III: 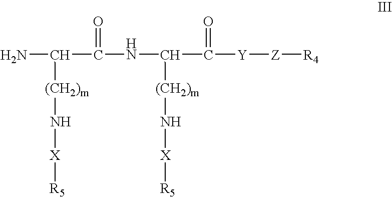
wherein m is from 1 to about 10.

The HBGF analog of formula III thus further includes a HBGF analog of formula IV: 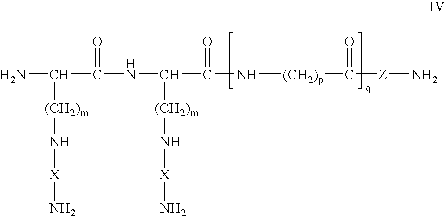
wherein p is from 1 to about 10 and q is from 1 to about 20. In one particularly preferred embodiment, p is 5 and q is three.

In one embodiment, in the HBGF analog of any of formula I or II where n=1, or of formula III or IV, the peptide chains X are cross-linked or cyclized. Such cross-linking or cyclization may be through a covalent bond, including at least one disulfide, peptide, amide or thioether bond.

In another embodiment, in the HBGF analog of any of formula I, II or III, Y includes between one and about thirty-three ethylene glycol (oxyethylene) units. Alternatively, Y may include a hydrophobic branched or unbranched, saturated or unsaturated alkyl chain of between one and about twenty carbon atoms. In a particularly preferred embodiment, Y is [NH₂-(CH₂)ₚ(C|O)]_{q} wherein p is from 1 to about 10 and q is from 1 to about 20. In another embodiment, Y includes a peptide sequence, and in a preferred embodiment, with from one to about 16 Gly residues.

In another embodiment of the HBGF analog of any of formula I, II, III or IV, each heparin binding motif of Z is BxBB, or BBBxxB, wherein each B independently represents lysine, arginine, ornithine, or histidine, and x represents a naturally occurring amino acid. In a preferred embodiment, Z includes at least two heparin-binding motifs, more preferably at least five heparin-binding motifs.

In another embodiment, the HBGF analog may be a molecule comprising two different amino acid residues each binding a different HBGFR, a hydrophobic linker region and a heparin-binding region. Thus the HBGF analog employed in this invention may one of formula V: 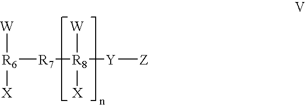
wherein each X and each W is a peptide chain differing by at least one amino acid residue that (i) has a minimum of three amino acid residues, (ii) has a maximum of about fifty amino acid residues, and (iii) binds a heparin-binding growth factor receptor (HBGFR); R₆ is a trifunctional amino acid residue covalently bonded to W and X or a dipeptide of the formula AA₁-AA₂; R₇ is a linker comprising a chain from 3 to about 20 atoms covalently bonded to R₆ and Y when n=0, or to R₆ and R₈ when n=1; R₈ is a dipeptide of the formula AA₂-AA₂; Y is a linker as defined above; Z is a non-signaling peptide chain that includes a heparin binding domain as defined above. Where provided, AA₁ is an amino acid residue, wherein one of X or W is covalently bonded through the N-terminus of AA₁ or through a side chain of AA₁; AA₂ is in each instance independently a trifunctional amino acid residue, wherein one of X or W is covalently bonded through a side chain of AA₂; and, n is 0 or 1.

In a preferred embodiment of the heparin-binding growth factor analog of formula V, X, W and Z are synthetic peptide chains. In the HBGF analog of formula V, Y can further consist of a linker that (i) is hydrophobic, (ii) comprises a chain of a minimum of about 9 and a maximum of about 50 atoms, and (iii) is not found in the natural ligand of the heparin-binding growth factor receptor (HBGFR) which X or W binds. In one embodiment of the HBGF analog of formula I, R₆ is a trifunctional amino acid residue, wherein X is covalently bonded through a side chain of R₆. The HBGF analog of formula V may be characterized, in certain embodiments, as having an avidity for heparin such that the HBGF analog binds heparin in 0.15 M NaCl, but is eluted by 1 M NaCl.

In another embodiment, the invention provides a HBGF of formula VI: 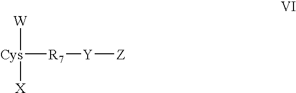
wherein Cys is cysteine; R₇ is a linker consisting of a sulfhydryl reactive homo-bifunctional cross-linker and a second Cys or comprising a hetero-bifunctional cross-linker; and W, X, Y and Z are as defined for formula V.

In yet another embodiment, the HBGF analog may be a construct of formula VII: 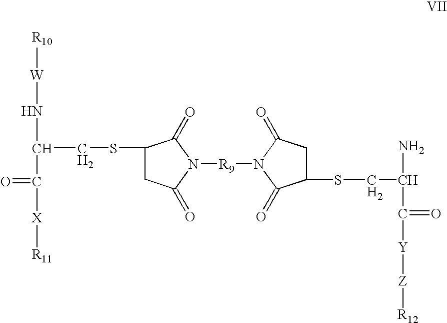
wherein R₉ is a linker comprising a chain of between 1 and about 10 backbone atoms selected from carbon, oxygen, sulfur and nitrogen or mixtures thereof; R₁₀ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative; R₁₁ is OH, NH₂, or NH—R₁₀; R₁₂ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative; and W, X, Y and Z are as defined for formula V.

In yet another embodiment, the HBGF may be a construct of formula VIII:

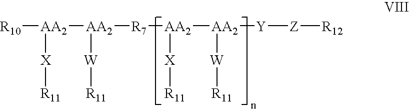

wherein R₇, R₁₀, R₁₁, and R₁₂ are as defined above; AA₂ is in each instance independently a trifunctional amino acid residue, wherein each X or W is covalently bonded through a side chain of AA₂; and X, W, Y and Z are as defined in formula V.

In the constructs of formulas V, VI or VIII, AA₂ may be in each instance a diamine amino acid residue, including a 2,3 diamino propionyl amino acid residue, a 2,4 diamino butylic amino acid residue, lysine or ornithine.

In the HBGF analogs of formula V, covalent bonds between R₆ and R₇ and between R₇ and Z when n=0, or between R₈ and Z when n=1, can be an amide, disulfide, thioether, Schiff base, reduced Schiff base, imide, secondary amine, carbonyl, urea, hydrazone or oxime bond.

In one embodiment of the HBGF of formulas V, VI or VIII, the side chains of AA₁ and AA₂ can include reactive carboxyl groups.

In another embodiment, the HBGF analog may be a molecule comprising two identical amino acid residues binding a HBGFR, but separated by a spacer sequence, and further with a hydrophobic linker region and a heparin-binding region. Thus the HBGF analog employed in this invention may one of formula IX: 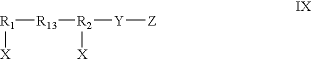
wherein each X is a peptide chain that (i) has a minimum of three amino acid residues, (ii) has a maximum of about fifty amino acid residues, and (iii) binds a heparin-binding growth factor receptor (HBGFR); R₁ is an amino acid residue, wherein X is covalently bonded through the N-terminus of R₁ or through a side chain of R₁; R₁₃ is a linker comprising a chain from 3 to about 20 backbone atoms covalently bonded to R₁ and R₂; R₂ is a trifunctional alpha amino acid residue, wherein X is covalently bonded through a side chain of R₂; Y is a linker comprising a chain from 0 to about 50 backbone atoms covalently bonded to R₂ and Z; and Z is a non-signaling peptide chain that comprises a heparin binding domain, comprising an amino acid sequence that comprises (i) a minimum of one heparin binding motif, (ii) a maximum of about ten heparin binding motifs.

In the HBGF analog of formula IX, R₁₃ can further include a linker that (i) comprises a chain of a minimum of about 3 and a maximum of about 20 atoms, and (ii) is not found in the natural ligand of the HBGFR which X binds. R₁₃ can further include a linker comprising a repeat unit, such as for example amino hexanoic acid (Hex) repeat units or amino acid repeat units, such as Gly repeats.

In the HBGF analog of formula IX, Y can further include a linker that (i) is hydrophobic, (ii) comprises a chain of a minimum of about 9 and a maximum of about 50 atoms, and (iii) is not found in the natural ligand of the HBGFR which X binds. In one embodiment of formula IX, R₁ is a trifunctional amino acid residue, wherein X is covalently bonded through a side chain of R₁.

In one embodiment of formula IX, the HBGF analog of formula IX is characterized in that it has an avidity for heparin such that the it binds heparin in 0.15 M NaCl, but is eluted by 1 M NaCl.

In another embodiment, the invention provides an HBGF analog of formula X: 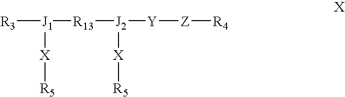
wherein R₃ and R₅ are each independently NH₂, an acyl group with a linear or branched C₁ to C₁₇ alkyl, aryl, heteroaryl, alkene, alkenyl or aralkyl chain including a N-terminus NH₂, NH₃⁺, or NH group or a corresponding acylated derivative, or is an amino acid, a dipeptide or a tripeptide, with an N-terminus NH₂, NH₃⁺, NH group or a corresponding acylated derivative; R₄ is —OH, NH₂, NH—R₁₄, or is an amino acid, a dipeptide or a tripeptide with a C-terminus —OH, NH₂, or NH—R₁₄; R₁₄ is an aliphatic C₁ to C₁₇ chain; each X is a peptide chain that (i) has a minimum of three amino acid residues, (ii) has a maximum of about fifty amino acid residues, and (iii) binds a HBGFR; J₁ and J₂ are each independently a trifunctional alpha amino acid residue, wherein each X is covalently bonded through a side chain of J₁ or J₂; R₁₃ is a linker comprising a chain from 3 to about 20 backbone atoms covalently bonded to J₁ and J₂; Y is a linker comprising a chain from 0 to about 50 backbone atoms covalently bonded to J₂ and Z; and Z is a non-signaling peptide that comprises a heparin binding domain, comprising an amino acid sequence that comprises (i) a minimum of one heparin binding motif, (ii) a maximum of about ten heparin binding motifs, and (iii) a maximum of about thirty amino acids.

In the HBGF analog of formula X, R₁₃ can further include a linker that (i) comprises a chain of a minimum of about 1 and a maximum of about 20 atoms, and (ii) is not found in the natural ligand of the HBGFR which X binds. R₁₃ can further include a linker comprising a repeat unit, such as for example Hex repeat units or amino acid repeat units, such as Gly repeats.

In the HGBF analog of formula X, Y further preferably includes a linker that (i) hydrophobic, (ii) comprises a chain of a minimum of about 9 and a maximum of about 50 atoms, and (iii) is not found in the natural ligand of the HBGFR which X binds.

In one embodiment, the HBGF analog of formula X is characterized in that it has an avidity for heparin such that it binds heparin in 0.15 M NaCl, but is eluted by 1 M NaCl.

The HBGF analog of formula X can further be characterized in that binding of it to the HBGFR initiates a signal by the HBGFR, or alternatively in that it blocks signaling by the HBGFR.

In one embodiment of the HBGF analog of formula X, J₁ and, if n=1, J₂, is a diamine amino acid residue. Such diamine amino acid residue may be a 2,3 diamino propionyl amino acid residue, a 2,4 diamino butylic amino acid residue, a lysyl residue or an ornithinyl residue. In an alternative embodiment of the HBGF analog of formula II, the side chain of J₁ and, if n=1, J₂, includes a reactive carboxyl group.

In one embodiment of the HBGF analog of formula X, the covalent bond between X and J₁ or, if n=1, J₂, comprises an amide, disulfide, thioether, Schiff base, reduced Schiff base, imide, secondary amine, carbonyl, urea, hydrazone or oxime bond. In a preferred embodiment, the bond is an amide bond.

The HBGF analog of formula II thus further includes a HBGF analog of formula XI: 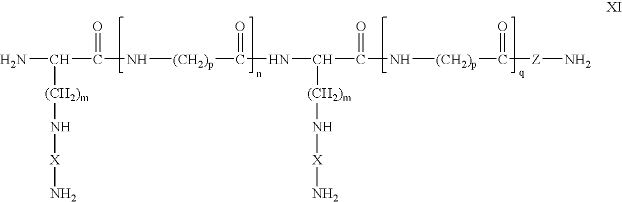
wherein each m is independently from 1 to about 10, each p is independently from 1 to about 10, q is from 1 to about 20, and n is from 1 to about 6. In one particularly preferred embodiment, p is 5, q is 3, m is 4, and n is 3.

In one embodiment, in the HBGF analog of the foregoing formulas, the peptide chains X are cross-linked or cyclized. Such cross-linking or cyclization may be through a covalent bond, including at least one disulfide, peptide, amide or thioether bond. In another embodiment, the terminal amines of each X, if provided, are crosslinked by means of a dialdehyde or by conjugation with a bifunctional crosslinking agent containing maleimides, aldehydes, succinymidal esters, benzotriazole carbonate, para-nitrophenol, or the like.

In another embodiment, the HBGF analog of any of the foregoing formulas includes between one and about thirty-three ethylene glycol (oxyethylene) units. Alternatively, Y may include a branched or unbranched, saturated or unsaturated alkyl chain of between one and about twenty carbon atoms. In a particularly preferred embodiment, Y is [NH₂-(CH₂)ₚ(C|O)]_{q} wherein p is from 1 to about 10 and q is from 1 to about 20. In another embodiment, Y includes a peptide sequence, and in a preferred embodiment, with from one to about 16 Gly residues.

The Heparin-Binding Growth Factors of the Foregoing Formulas

The regions X and Z of the synthetic HBGF analogs of the foregoing formulas include amino acid residues, and optionally the region Y includes amino acid residues. An amino acid residue is defined as —NHRCO—, where R can be hydrogen or any organic group. The amino acids can be D-amino acids or L-amino acids. Additionally, the amino acids can be α-amino acids, β-amino acids, γ-amino acids, or δ-amino acids and so on, depending on the length of the carbon chain of the amino acid.

The amino acids of the X, Y and Z component regions of the synthetic HBGF analogs of the invention can include any of the twenty amino acids found naturally in proteins, i.e. alanine (Ala, A), arginine (Arg, R), asparagine (Asn, N), aspartic acid (Asp, D), cysteine (Cys, C), glutamic acid (Glu, E), glutamine (Gln, Q), glycine (Gly, G), histidine (His, H), isoleucine, (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V).

Furthermore, the amino acids of the X, Y and Z component regions of the synthetic HBGF analogs of the invention can include any of the naturally occurring amino acids not found naturally in proteins, e.g. β-alanine, betaine (N,N,N-trimethylglycine), homoserine, homocysteine, γ-amino butyric acid, ornithine, and citrulline.

Additionally, the amino acids of the X, Y and Z component regions of the synthetic HBGF analogs of the invention can include any of the non-biological amino acids, i.e. those not normally found in living systems, such as for instance, a straight chain amino-carboxylic acid not found in nature. Examples of straight chain amino-carboxylic acids not found in nature include 6-aminohexanoic acid, and 7-aminoheptanoic acid, 9-aminononanoic acid and the like.

The amino acid R₁ of formula I or any of the foregoing formulas can be any of the amino acids described above. R₂ of formula I or any of the foregoing formulas, and J₁ and J₂ of formula II or any of the foregoing formulas, can be any trifunctional amino acid residue, preferably a trifunctional alpha amino acid residue. In a preferred embodiment, the trifunctional amino acid residue is a diamine amino acid, such as for instance lysine or ornithine, or any other amino acid having two amino groups.

The region X and, if provided, the region W of the foregoing formulas of synthetic HBGF analogs is a synthetic peptide chain that binds an HBGFR. Region X or W can, for example, have any amino acid sequence that binds an HBGFR, and can include amino acid sequences that are identical to a portion of the amino acid sequence of a HBGF. Alternatively, X or W can have an amino acid sequence homologous rather than identical to the amino acid sequence of an HBGF. The particular HBGFR bound by the synthetic HBGF analog of the invention may or may not be the cognate receptor of the original HBGF, i.e. the synthetic HBGF analog may additionally or solely bind to the receptor of a different HBGF.

The region W of the foregoing formulas of synthetic HBGF analogs is a synthetic peptide chain that binds an HBGFR and is different from the region X. Region X or W can, for example, each independently have any amino acid sequence that binds an HBGFR, and can include amino acid sequences that are identical to a portion of the amino acid sequence of a HBGF. Alternatively, X or W can each independently have an amino acid sequence homologous rather than identical to the amino acid sequence of an HBGF. The particular HBGFR bound by the synthetic HBGF analog of the invention may or may not be the cognate receptor of the original HBGF, i.e. the synthetic HBGF analog may additionally or solely bind to the receptor of a different HBGF.

The term ‘homologous’, as used herein refers to peptides that differ in amino acid sequence at one or more amino acid positions when the sequences are aligned. For example, the amino acid sequences of two homologous peptides can differ only by one amino acid residue within the aligned amino acid sequences of five to ten amino acids. Alternatively, two homologous peptides of ten to fifteen amino acids can differ by no more than two amino acid residues when aligned. In another alternative, two homologous peptides of fifteen to twenty or more amino acids can differ by up to three amino acid residues when aligned. For longer peptides, homologous peptides can differ by up to approximately 5%, 10%, 20% or 25% of the amino acid residues when the amino acid sequences of the two peptide homologs are aligned.

Particularly useful amino acid sequences as X or W regions include homologs of fragments of naturally occurring HBGFs that differ from the amino acid sequences of natural growth factor in only one or two or a very few positions. Such sequences preferably include conservative changes, where the original amino acid is replaced with an amino acid of a similar character according to well known principles; for example, the replacement of a non-polar amino acid such as alanine with valine, leucine, isoleucine or proline; or the substitution of one acidic or basic amino acid with another of the same acidic or basic character.

In another alternative, the X or W region of the synthetic HBGF analog can include an amino acid sequence that shows no detectable homology to the amino acid sequence of any HBGF. Peptides or growth factor analogs useful as components of the X or W region of the synthetic analogs of the present invention, that have little or no amino acid sequence homology with the cognate growth factor and yet bind HBGFRs may be obtained by any of a wide range of methods, including for instance, selection by phage display. See as an example: Sidhu et al. Phage display for selection of novel binding peptides. Methods Enzymol. 328:333-63 (2000). An example of such a peptide that binds an HBGFR yet has no homology to any known HBGF is the C19 peptide sequence described below in Example 1.

The X or W region of the synthetic HBGF analogs can have any length that includes an amino acid sequence that effectively binds an HBGFR. Preferably, the X or W regions of the synthetic HBGF analogs have a minimum length of at least approximately three amino acid residues. More preferably, the X or W regions of the synthetic HBGF analogs have a minimum length of at least approximately six amino acid residues. Most preferably the X or W regions of the synthetic HBGF analogs have a minimum length of at least approximately ten amino acid residues. The X or W regions of the synthetic HBGF analogs of the invention preferably also have a maximum length of up to approximately fifty amino acid residues, more preferably a maximum length of up to approximately forty amino acid residues, and most preferably a maximum length of up to approximately thirty amino acid residues.

In one embodiment of the synthetic HBGF analogs that include two X or W regions, the X or W regions are covalently cross-linked. Suitable cross links can be formed by S-S bridges of cysteines linking the two X or W regions. Alternatively, the cross link can be conveniently formed during simultaneous and parallel peptide synthesis of the X or W region amino acids chains by incorporating a lanthionine (thio-dialanine) residue to link the two identical X chains at alanine residues that are covalently bonded together by a thioether bond. In another method the two X or W region amino acid chains can be cross-linked by introducing a cross-linking agent, such as a dicarboxylic acid, e.g. suberic acid (octanedioic acid), or the like, thereby introducing a hydrocarbon bridge between the two identical X or W regions having a free amino, hydroxyl or thiol group. The cross-linked X or W regions can constitute a cyclic peptide, such as where the terminal amino acids of X are cross-linked through reactive side chains or the terminal groups, optionally with a bridge or other link. An X or W region and a W region may be similarly cross-linked.

In the synthetic HBGF analogs, the Y region of the foregoing formulas is a linker that is sufficiently hydrophobic to non-covalently bind the HBGF analog to a medical device surface, including a polymeric or metal medical device surface. Such surfaces are typically hydrophobic surfaces. Examples of suitable surfaces include but are not limited to those formed from hydrophobic polymers such as polycarbonate, polyester, polypropylene, polyethylene, polystyrene, polytetrafluoroethylene, expanded polytetrafluoroethylene, polyvinyl chloride, polyamide, polyacrylate, polyurethane, polyvinyl alcohol, polyurethane, poly ethyl vinyl acetate, poly(butyl methacrylate), poly(ethylene-co-vinyl acetate), polycaprolactone, polylactide, polyglycolide and copolymers of any two or more of the foregoing; siloxanes such as 2,4,6,8-tetramethylcyclotetrasiloxane; natural and artificial rubbers; glass; and metals including stainless steel, titanium, platinum, and nitinol. Preferably, the binding of the HBGF analogs to the hydrophobic surface is of sufficient quantity to be detected by an analytical method such as an enzyme-linked immunoassay or a biological assay.

The Y region of the foregoing formulas can include a chain of atoms or a combination of atoms that form a chain. Typically, the chains are chains of carbon atoms, that may also optionally include oxygen, nitrogen or sulfur atoms, such as for example chains of atoms formed from amino acids (e.g. amino acids found in proteins, as listed above; naturally occurring amino acids not found in proteins, such as ornithine and citrulline; or non natural amino acids, such as amino hexanoic acid; or a combination of any of the foregoing amino acids).

The chain of atoms of the Y region can, in certain of the foregoing formulas, be attached to R₁ or R₂ and to peptide Z. Similarly the chain of atoms of the Y region of certain of the foregoing formulas can be covalently attached to J₁ or J₂ and to peptide Z. The covalent bonds can be, for example, peptide, amide or ester bonds. Preferably, the Y region includes a chain of a minimum of about nine atoms. More preferably, the Y region includes a chain of a minimum of about twelve atoms. Most preferably, the Y region includes a chain of a minimum of about fifteen atoms. For example, the Y region may be formed from a chain of at least four, at least five or at least six amino acids. Alternatively, the Y region may be formed from a chain of at least one, at least two, or at least three aminohexanoic acid residues.

Preferably, the Y region includes a chain of a maximum of about fifty atoms. More preferably, the Y region includes a chain of a maximum of about forty-five atoms. Most preferably, the Y region includes a chain of a maximum of about thirty-five atoms. For example, the Y region may be formed from a chain of up to about twelve, up to about fifteen, or up to about seventeen amino acids.

The amino acid sequence of the Y region is preferably an artificial sequence, i.e. it does not include any amino acid sequence of four or more amino acid residues found in a natural ligand of a HBGF.

In a particular embodiment, the Y region includes a hydrophobic amino acid residue, or a chain of hydrophobic amino acid residues. The Y region can, for example, include one or more aminohexanoic acid residues, such as one, two, three or more aminohexanoic acid residues.

In another particular embodiment, the Y region of the molecule can include a hydrophobic branched or unbranched, saturated or unsaturated alkyl chain of between one and about twenty carbon atoms. In a further embodiment, the Y region can include a chain of hydrophobic residues, such as for instance, ethylene glycol residues. For instance, the Y region can include at least about three, or at least about four, or at least about five ethylene glycol residues. Alternatively, the Y region can include up to about twelve, up to about fifteen, or up to about seventeen ethylene glycol residues. In another alternative embodiment, the Y region can include a combination of amino acid hydrophobic residues.

The Z region of the foregoing formulas is a heparin-binding region and can include one or more heparin-binding motifs, BBxB or BBBxxB as described by Verrecchio et al. J. Biol. Chem. 275:7701 (2000). Alternatively, the Z region can include both BBxB and BBBxxB motifs (where B represents lysine, arginine, or histidine, and x represents a naturally occurring, or a non-naturally occurring amino acid). For example, the heparin-binding motifs may be represented by the sequence [KR][KR][KR]X(2)[KR] (SEQ ID NO:1), designating the first three amino acids as each independently selected from lysine or arginine, followed by any two amino acids and a sixth amino acid which is lysine or arginine.

The number of heparin binding motifs is variable. For instance, the Z region may include at least one, at least two, at least three or at least five heparin-binding motifs. Where there are more than one heparin-binding motifs, the motifs may be the same or different. Alternatively, the Z region includes up to a maximum of about ten heparin-binding motifs. In another alternative embodiment, the Z region includes at least four, at least six or at least eight amino acid residues. Further, in certain embodiments the Z region includes up to about twenty, up to about, twenty-five, or up to about thirty amino acid residues. It is to be realized that, in part, the avidity of the Z region for heparin is determined by the particular heparin-binding motifs selected and the number of such motifs in Z. Thus for particular applications both the selection and number of such motifs may be varied to provide optimal heparin binding of the Z region.

In a preferred embodiment, the amino acid sequence of the Z region is RKRKLERIAR (SEQ ID NO:2). In another embodiment, the amino acid sequence of the Z region is RKRKLGRIAR (SEQ ID NO:3). In yet another embodiment, the amino acid sequence of the Z region is RKRKLWRARA (SEQ ID NO:4). In yet another embodiment, the amino acid sequence of the Z region is RKRLDRIAR (SEQ ID NO:5). In yet another embodiment, the amino acid sequence of the Z region is RKRKLERIARC (SEQ ID NO:6). The presence of a terminal cysteine residue optionally affords the opportunity to link other molecules, including detection reagents such as fluorochromes, radioisotopes and other detectable markers, to the Z region, as well as the opportunity to link toxins, immunogens and the like.

Heparin-binding domains that bear little or no sequence homology to known heparin-binding domains are also contemplated in the present invention. As used herein the term “heparin-binding” means binding to the —NHSO₃⁻ and sulfate-modified polysaccharide, heparin, and also binding to the related modified polysaccharide, heparan sulfate, as well as glycosaminoglycans or proteoglycans containing heparin or heparin sulfate, as well as degration products of the aforementioned, and synthetic polysaccharides that exhibit heparin-like activity.

The Z region of the synthetic HBGF analogs of the present invention confers the property of binding to heparin in low salt concentrations, up to about 0.15 M NaCl, optionally up to about 0.48 M NaCl, forming a complex between heparin and the Z region of the factor analog. The complex can be dissociated in 1 M NaCl to release the synthetic HBGF analog from the heparin complex.

The Z region is a non-signaling peptide relative to the HBGFR. Accordingly, when used alone the Z region binds to heparin which can be bound to a receptor of a HBGF, but the binding of the Z region peptide alone does not initiate or block signaling by the receptor.

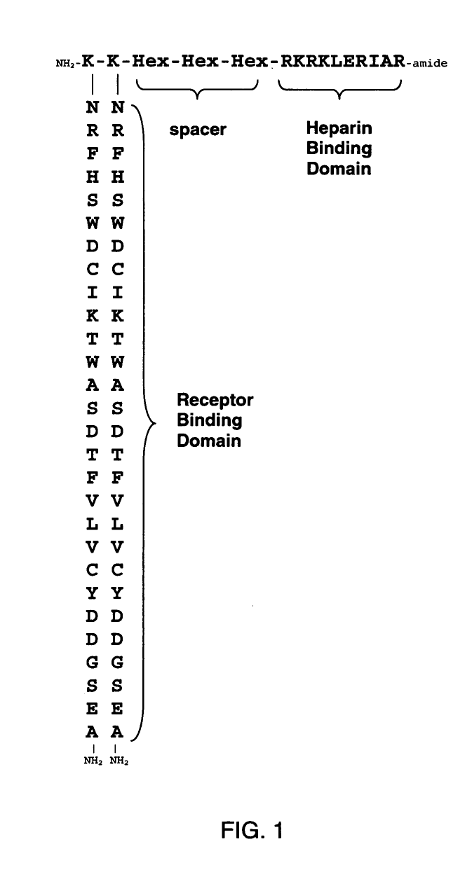

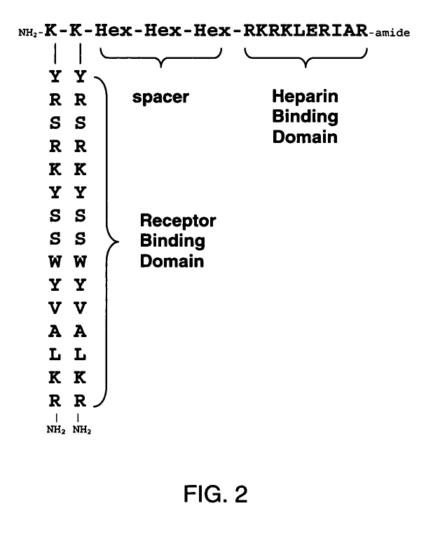

The C-terminus of the Z region may be blocked or free. For example, the C terminus of the Z region may be the free carboxyl group of the terminal amino acid, or alternatively, the C terminus of the Z region may be a blocked carboxyl group, such as for instance, an amide group. In a preferred embodiment the C terminus of the Z region is an amidated arginine as shown in FIGS. 1 and 2.

As used here and elsewhere, the following terms have the meanings given.

The term “alkene” includes unsaturated hydrocarbons that contain one or more double carbon-carbon bonds. Examples of such alkene groups include ethylene, propene, and the like.

The term “alkenyl” includes a linear monovalent hydrocarbon radical of two to six carbon atoms or a branched monovalent hydrocarbon radical of three to six carbon atoms containing at least one double bond; examples thereof include ethenyl, 2-propenyl, and the like.

The “alkyl” groups specified herein include those alkyl radicals of the designated length in either a straight or branched configuration. Examples of such alkyl radicals include methyl, ethyl, propyl, isopropyl, butyl, sec-butyl, tertiary butyl, pentyl, isopentyl, hexyl, isohexyl, and the like.

The term “aryl” includes a monovalent or bicyclic aromatic hydrocarbon radical of 6 to 12 ring atoms, and optionally substituted independently with one or more substituents selected from alkyl, haloalkyl, cycloalkyl, alkoxy, alkythio, halo, nitro, acyl, cyano, amino, monosubstituted amino, disubstituted amino, hydroxy, carboxy, or alkoxy-carbonyl. Examples of an aryl group include phenyl, biphenyl, naphthyl, 1-naphthyl, and 2-naphthyl, derivatives thereof, and the like.

The term “aralkyl” includes a radical—R^{a}R^{b} where R^{a} is an alkylene (a bivalent alkyl) group and R^{b} is an aryl group as defined above. Examples of aralkyl groups include benzyl, phenylethyl, 3-(3-chlorophenyl)-2-methylpentyl, and the like. The term “aliphatic” includes compounds with hydrocarbon chains, such as for example alkanes, alkenes, alkynes, and derivatives thereof.

The term “acyl” includes a group RCO—, where R is an organic group. An example is the acetyl group CH₃CO—.

A peptide or aliphatic moiety is “acylated” when an alkyl or substituted alkyl group as defined above is bonded through one or more carbonyl {—(C|O)—} groups. A peptide is most usually acylated at the N-terminus.

An “amide” includes compounds that have a trivalent nitrogen attached to a carbonyl group (—CO.NH₂).

An “amine” includes compounds that contain an amino group (—NH₂).

“Heparin” as used herein includes heparin, low-molecular-weight variants thereof, or fragments thereof, or any of a number of compounds that bind growth factors in a manner similar to heparin. Such compounds include but are not limited to heparan sulfate, chondroitin sulfate, hyaluronic acid, dextran sulfate, carboxymethyl cellulose, or any of a number of synthetic heparin-mimicking polyanionic compounds. “Heparin” also includes but is not limited to molecules including a mixture of variably sulfated polysaccharide chains composed of repeating units of d-glucosamine and either l-iduronic or d-glucuronic acids, salts of any of the foregoing and derivatives of any of the foregoing. For example, conventional salts of heparin include sodium heparin, calcium heparin, magnesium heparin, and potassium heparin. Heparin derivatives include, but are not limited to ammonium heparin, benzalkonium heparin, and the like. Heparin further includes silyl-heparin compositions as described in U.S. patent application Ser. No. 10/450,309, which is U.S. Patent Application Publication No. 2004/0161442 A1, entitled “Bioactive Coating Compositions and Methods”, to Paul O. Zamora, et al., filed on Jan. 28, 2003, the specification of which is hereby incorporated by reference.

FGF Synthetic Analogs

In another particular aspect, the invention provides a synthetic FGF peptide analog used in a coating or method of coating of the invention. The synthetic FGF analogs represented by any of the foregoing formulas, wherein X, or W if it is provided, is an FGF analog which can be any FGF, such as any of the known FGFs, including all 23 FGFs from FGF-1 to FGF-23.

The X or W region of the molecule of formulas of the present invention can include an amino acid sequences found in an FGF, such as for instance FGF-2 or FGF-7. Alternatively, the X or W regions can include sequences not found in the natural ligand of the FGFR bound by the molecule.

The X or W region of synthetic FGF peptide analogs can include an amino acid sequence that is 100% identical to an amino acid sequence found in a fibroblast growth factor or an amino acid sequence homologous to the amino acid sequence of a fibroblast growth factor. For instance, the X or W region can include an amino acid sequence that is at least about 50%, at least about 75%, or at least about 90% homologous to an amino acid sequence from a fibroblast growth factor. The fibroblast growth factor can be any fibroblast growth factor, including any of the known or yet to be identified fibroblast growth factors.

In a particular embodiment, the synthetic FGF analog of the invention is an agonist of the HBGFR. When bound to the HBGFR, the synthetic HBGF analog initiates a signal by the HBGFR.

In a further particular embodiment, the synthetic FGF analog of the invention is an antagonist of the HBGFR. When bound to the HBGFR, the synthetic HBGF analog blocks signaling by the HBGFR.

In another particular embodiment of the present invention, the synthetic FGF analog is an analog of FGF-2 (also known as basic FGF, or bFGF). In another particular embodiment of the present invention, the binding of the synthetic FGF analog to an FGF receptor initiates a signal by the FGF receptor. In a further particular embodiment, the binding of the synthetic FGF analog to the FGF receptor blocks signaling by the FGF receptor.

In a yet further particular embodiment, the present invention provides a synthetic FGF analog of FGF-2. In another particular embodiment, the present invention provides a synthetic FGF analog of FGF-2, wherein the amino acid sequence of the X or W region is YRSRKYTSWYVALKR (SEQ ID NO:7) from FGF-2. In yet another particular embodiment, the present invention provides a synthetic FGF analog wherein the amino acid sequence of the X or W region is NRFHSWDCIKTWASDTFVLVCYDDGSEA (SEQ ID NO:8). In yet another particular embodiment, the present invention provides a synthetic FGF-2 analog wherein the amino acid sequence of the X or W region is HIKLQLQAEERGWS (SEQ ID NO:9).

In a yet further particular embodiment, the invention provides a synthetic FGF analog of FGF-1, wherein the X or W region is YISKKHAEKNWFVGLKK (SEQ ID NO:10). This sequence is derived from amino acids bridging the beta 9 and beta 10 loop of FGF-1. In yet another particular embodiment, an FGF-1 analog is provided wherein the X or W region is HIQLQLSAESVGEVY (SEQ ID NO:11), corresponding to amino acids derived from the β-4 and β-5 region of FGF-1.

In a yet further particular embodiment, the invention provides a synthetic FGF analog of FGF-7, wherein the X or W region is YASAKWTHNGGEMFVALNQK (SEQ ID NO:12). In yet another embodiment of a synthetic FGF analog of FGF-7, the X or W region is the amino acid sequence YNIMEIRTVAVGIVA (SEQ ID NO:13).

Other FGF receptor binding domains, derived largely from targeting sequences in the C-terminus of human FGF, include the following sequences shown in Table 1:

TABLE 1

| | |
|---|---|
| CYTOKINE | PREFERRED X OR W RECEPTOR BINDING DOMAIN |

| | | |
|---|---|---|
| FGF-10 | YASFNWQHNGRQMYVALNQK | (SEQ ID NO:14) |
| FGF-22 | YASQRWRRRGQPNLALDRR | (SEQ ID NO:15) |
| FGF-9 | YSSNLYKHVDTGRRYYVALNK | (SEQ ID NO:16) |
| FGF-16 | YASTLYKHSDSERQYVALNK | (SEQ ID NO:17) |
| FGF-20 | YSSNIYKHGDTGRRFVALNK | (SEQ ID NO:18) |
| FGF-4 | YESYKYPGMFIALSKN | (SEQ ID NO:19) |
| FGF-6 | YESDLYQGTYILSKYGR | (SEQ ID NO:20) |
| FGF-12 | YSSTLYRQQESGRAWFLGNK | (SEQ ID NO:21) |
| FGF-14 | YSSMLYRQQESGRAWFLGLNK | (SEQ ID NO:22) |
| FGF-13 | YSSMIYRQQQSGRGWYLGLNK | (SEQ ID NO:23) |
| FGF-11 | YASALYRQRRSGRAWYLDK | (SEQ ID NO:24) |

Table 2 below compares the actions of two synthetic FGF analogs, F2A3 and F2A4, with that of recombinant FGF-2. In growth studies, the specific cell lines that were used included murine C3H10T1/2 fibroblasts, A7R5 murine smooth muscle cells, human umbilical vein endothelial cells (HUVEC), bovine aorta endothelial cells (BAE), rat microvascular endothelial cells (RMEC), and CG4 glioma cells. Changes in smooth muscle actin and TGF-β RII (receptor for transforming growth factor-beta) were monitored by immunochemistry. Nitric oxide (NO) production was monitored by fluorescence microscopy using 2,4-diaminofluorescein as the fluorogen. Angiogenesis was monitored following introduction of coated sutures in rat muscle. Salivary gland stimulation was determined by monitoring saliva production. Wound healing was monitored in full thickness wounds of rat skin. In Table 2 and hereafter, “N.D.” means not determined.

TABLE 2

| | | |
|---|---|---|
| FGF-2 | F2A3 | F2A4 |

Biochemical

| | | | |
|---|---|---|---|
| Interaction with heparin | Yes | Yes | Yes |
| Binding to FGF receptor | Yes | Yes | Yes |
| Binding to VEGF receptor | No | No | No |
| MAP kinase phosphorylation and activation | Yes | Yes | Yes |

Growth stimulation

| | | | |
|---|---|---|---|
| Fibroblasts, Endothelial cells, Smooth | Yes | Yes | Yes |
| muscle cells, Neural cells | | | |

Cellular changes

| | | | |
|---|---|---|---|
| Increased smooth muscle cell actin | Yes | Yes | Yes |
| Increased NO production, endothelial cells | Yes | Yes | N.D. |
| Decreased TGF-β RII, endothelial cells | Yes | Yes | N.D. |
| Radiation protection | Yes | Yes | Yes |

In vivo

| | | | |
|---|---|---|---|
| Angiogenisis | Yes | Yes | Yes |
| Radiation protection, in vivo | Yes | Yes | Yes |
| Salivary gland stimulation | Yes | Yes | N.D. |
| Accelerated skin wound healing | Yes | Yes | N.D. |

VEGF Synthetic Analogs

In another particular aspect, the invention provides a synthetic VEGF peptide analog. The synthetic VEGF analogs represented include, in one embodiment, a VEGF analog wherein the amino acid sequence of the X or W region is APMAEGGGQNHHEWKFMDV (SEQ ID NO:25). In another embodiment, there is provided a synthetic VEGF peptide analog wherein the amino acid sequence of the X or W region is GATWLPPNPTK (SEQ ID NO:26). In yet another embodiment, there is provided a synthetic VEGF peptide analog wherein the amino acid sequence of the X or W region is NFLLSWVHWSLALLLYLHHA (SEQ ID NO:27).

Table 3 below compares the actions of two synthetic VEGF peptide analogs, VA01 and VA02, with that of recombinant VEGF. For MAP kinase, bovine aorta endothelial (BAE) cells were stimulated with 50 ng/mL of VEGF, VA01 or VA02 for 30 or 60 minutes. Cell lysate was analysed by Western blotting using monoclonal anti-phospho-44/42 MAP kinase antibody (Thr202 and Tyr204) and increased phosphorylization of ERK-1 and ERK-2 relative to controls was detected following stimulation with VEGF, VA01 or VA02. For growth, an increase in relative cell number of BAE cells was found following stimulation with VEGF, VA01 or VA02 whereas in A7R5, a smooth muscle cell line, no growth stimulation was found, indicating specificity in the action of the analogs.

TABLE 3

| | | |
|---|---|---|
| VEGF | VA01 | VA01 |

Biochemical

| | | | |
|---|---|---|---|
| Interaction with heparin | Yes | Yes | Yes |
| MAP kinase phosphorylation | Yes | Yes | Yes |

Growth stimulation

| | | | |
|---|---|---|---|
| Endothelial cells | Yes | Yes | Yes |
| Smooth muscle cells | No | No | No |

Cellular changes

| | | | |
|---|---|---|---|
| Tube formation in collagen gels (in vitro | Yes | Yes | Yes |
| model of angiogenisis) | | | |

BMP Synthetic Analogs

In another particular aspect, the invention utilizes a synthetic BMP peptide analog. The synthetic bone morphogenic protein analogs include embodiments wherein the X or W region includes the amino acid sequence LYVDFSDVGWNDW (SEQ ID NO:28), AISMLYLDENEKWL (SEQ ID NO:29), ISMLYLDENEKVVLKNY (SEQ ID NO:30), EKWLKNYQDMWEG (SEQ ID NO:31), LWKENEDLYLMSIAC (SEQ ID NO:32), AFYCHGECPFPLADHL (SEQ ID NO:33), or PFPLADHLNSTNHAIVQTLVNSV (SEQ ID NO:34).

Alternatively, in another particular aspect the invention provides synthetic BMP, TGF or GDF (growth differentiation factor) peptide analogs as shown in Table 4 wherein the transforming growth factor family member peptides are particularly useful in augmenting the activity of endogenous or artificial BMP peptides or TGF peptides, wherein is shown (under the heading “preferred receptor binding domain”) the sequence forming all or part of the X or W region of constructs of any of the foregoing formulas.

TABLE 4

| | |
|---|---|
| CYTOKINE | PREFERRED X OR W RECEPTOR BINDING DOMAIN |

| | | |
|---|---|---|
| TGF-β1 | IVYYVGRKPKVEQLSNMIVRS | (SEQ ID NO:35) |
| TGF-β2 | TILYYIGKTPKIEQLSNMIVKS | (SEQ ID NO:36) |
| TGF-β3 | LTILYYVGRTPKVEQLSNMW | (SEQ ID NO:37) |
| BMP-2 | AISMLYLDENEKWLKNYQDMW | (SEQ ID NO:38) |
| BMP-3 | SSLSILFFDENKNWLKVYPNMTV | (SEQ ID NO:39) |
| BMP-3β | NSLGVLFLDENRNWLKVYPNMSV | (SEQ ID NO:40) |
| BMP-4 | AISMLYLDEYDKWLKNYQEMW | (SEQ ID NO:41) |
| BMP-5 | AISVLYFDDSSNVILKKYRNMW | (SEQ ID NO:42) |
| BMP-6 | AISVLYFDDNSNVILKKYRNMW | (SEQ ID NO:43) |
| BMP-7 | AISVLYFDDSSNVILKKYRNMW | ( SEQ ID NO:44) |
| BMP-8 | ATSVLYYDSSNNVILRKARNMW | (SEQ ID NO:45) |
| BMP-9 | ISVLYKDDMGVPTLKYHYEGMSV | (SEQ ID NO:46) |
| BMP-10 | ISILYLDKGVVTYKFKYEGMAV | (SEQ ID NO:47) |
| BMP-11 | INMLYFNDKQQIIYGKIPGMW | (SEQ ID NO:48) |
| BMP-12 | ISILYIDAANNWYKQYEDMW | (SEQ ID NO:49) |
| BMP-13 | ISILYIDAGNNWYKQYEDMW | (SEQ ID NO:50) |
| BMP-14 | ISILFIDSANNVVYKQYEDMW | (SEQ ID NO:51) |
| BMP-15 | ISVLMIEANGSILYKEYEGMIA | (SEQ ID NO:52) |
| GDF-1 | ISVLFFDNSDNWLRQYEDMW | (SEQ ID NO:53) |
| GDF-3 | ISMLYQDNNDNVILRHYEDMW | (SEQ ID NO:54) |
| GDF-8 | INMYLFNGKEQIIYGKIPAMW | (SEQ ID NO:55) |
| GDF-9 | LSVLTIEPDGSIAYKEYEDMIA | (SEQ ID NO:56) |

Table 5 below summarizes the bioechmical interactions of one BMP analog, B2A2, and the modulation of alkaline phosphatase, wherein modulation was monitored using C2C12 cells.

TABLE 5
Biochemical interactions of B2A2

| | |
|---|---|
| Interaction with heparin | Yes |
| MAP kinase phosphorylation | Yes |

Positive modulation of alkaline phosphatase

| | |
|---|---|
| BMP-2 (E. coli) | Yes |
| BMP-2 (Chinese hamster ovary cells) | Yes |
| BMP-7 (mammalian cell) | No |

Modulation via a coating of alkaline phosphatase

| | |
|---|---|
| B2A2 coating, BMP-2 in solution | Yes |
| BMP-2 coating, B2A2 in solution | Yes |
| Silyl-heparin/BMP-2 coating, B2A2 in solution | Yes |

Reverse Sequence X or W Regions

It has surprisingly and advantageously been found that in the compounds of the present invention, including those of foregoing formulas, the X or W region may be synthesized in a reverse direction, such that considering the sequence AISMLYLDENEKWL (SEQ ID NO:29) illustrated in the conventional N→C orientation, and using formula I, the first amino acid bound to either the R₁ side chain or N-terminus amine is the N-terminus amino acid residue (bound through its carboxyl group thereby forming a peptide bond), the second amino acid bound to the N-terminus amino acid residue is the 2 position residue, and so on, and the compounds nonetheless retain biological activity and specifically bind to a BMP receptor. It may be seen that such a construct has, based on a conventional N→C orientation, a reverse sequence, in that it is the carboxyl group of the conventional N-terminus amino acid residue that forms a peptide bond with an amine of R₁ where R₁ is a diamine amino acid. Thus again employing a conventional N→C orientation, the foregoing sequences may be employed in a reverse orientation, and the resulting compound of present invention is biologically active and may be employed as described herein. According to a preferred embodiment, the X or W region is the sequence LWKENEDLYLMSIA (SEQ ID NO:57) (again considering the sequence in the conventional N→C orientation.

Other reverse sequences that may be employed, in whole or in part, including homologs thereto, in addition to LVVKENEDLYLMSIA (SEQ ID NO:57), include but are not limited to YNKLWKENEDLYLMSI (SEQ ID NO:58), KKLIVNSSEDFYL (SEQ ID NO:59), WDNWGVDSFDVYL (SEQ ID NO:60), GEWMDQYNKLWKE (SEQ ID NO:61), LHDALPFPCEGHCYFA (SEQ ID NO:62), VSNVLTQVIAHNTSNLHDALPFP (SEQ ID NO:63), and LWKENEDLYLMSIAC (SEQ ID NO:64).

Reverse sequences may similarly be employed, in whole or in part, including homologs thereto, that are reverse sequences of FGF or other HBGF analogs. Thus a reverse sequence of SEQ ID NO:8 may be employed, which is the sequence AESGDDYCVLVFTDSAWTKICDWSHFRN (SEQ ID NO:65). Similarly, a reverse sequence of SEQ ID NO:7 may be employed, which is the sequence RKLAVYWSSYKRSRY (SEQ ID NO:66). A reverse sequence of SEQ ID NO:10 may also be employed, which is the sequence KKLGVFWNKEAHKKSIY (SEQ ID NO:67). A reverse sequence of SEQ ID NO:11 may also be employed, which is the sequence YVEGVESASLQLQIH (SEQ ID NO:68). A reverse sequence of SEQ ID NO:12 may also be employed, which is the sequence KQNLAVFMEGGNHTWKASAY (SEQ ID NO:69). A reverse sequence of SEQ ID NO:13 may also be employed, which is the sequence AVIGVAVTRIEMINY (SEQ ID NO:70).

Methods of Synthesizing the Heparin-Binding Growth Factor Analogs

The synthesis of the analogs of the invention can be achieved by any of a variety of chemical methods well known in the art. Such methods include bench scale solid phase synthesis and automated peptide synthesis in any one of the many commercially available peptide synthesizers. Preferably, the synthesizer has a per cycle coupling efficiency of greater than 99 percent.

The analogs of the present invention can be produced by stepwise synthesis or by synthesis of a series of fragments that can be coupled by similar well known techniques. See, for instance, Nyfeler, Peptide synthesis via fragment condensation. Methods Mol. Biol. 35:303-16 (1994); and Merrifield, Concept and early development of solid-phase peptide synthesis. Methods in Enzymol. 289:3-13 (1997). These methods are routinely used for the preparation of individual peptides. It is possible to assemble the analogs of the present invention in component parts, such as peptides constituting the X, W, Y and Z components thereof, and to thereafter couple such component parts to assemble the analog. See, for instance, Dawson and Kent, Synthesis of native proteins by chemical ligation. Annu. Rev. Biochem. 69:923-960 (2000); and Eom et al., Tandem ligation of multipartite peptides with cell-permeable activity. J. Am. Chem. Soc. 125:73-82 (2003).

Advantageously, in the case where the analogs of any of the foregoing formulas of the invention include two identical X region amino acid sequences, the synthesis of these identical X region peptides may be performed in parallel. By this method each cycle of addition adds an amino acid to both of the X region peptides, greatly facilitating the synthesis of these branched molecules.

Peptide libraries that can be used to screen for a desired property, such as binding to an HBGFR can be prepared by adaptations of these methods. See for instance, Fox, Multiple peptide synthesis, Mol. Biotechnol. 3:249-58 (1995); and Wade and Tregear, Solid phase peptide synthesis: recent advances and applications. Austral. Biotechnol. 3:332-6 (1993).

In a particular embodiment, the synthetic HBGF analog of the invention is an agonist of the HBGFR. When bound to the HBGFR, the synthetic HBGF analog initiates a signal by the HBGFR.

In another particular embodiment, the synthetic HBGF analog of the invention is an antagonist of the HBGFR. When bound to the HBGFR, the synthetic HBGF analog blocks signaling by the HBGFR.

In a particular aspect, the invention provides a method for stimulating growth factor receptor signaling in a cell by contacting the cell with an effective amount of a synthetic HBGF analog according to the foregoing formulas. The effective amount can be readily determined by one of skill in the art. The signaling can result in cytokine release from the cell, stimulation or inhibition of proliferation or differentiation of the cell, chemotaxis of the cell, stimulation or inhibition of the immune system of the mammal.

Methods of Use of the HBGFs of the Invention

The synthetic HBGF analogs of present invention are employed as biologically active agents for coating of medical devices, such as for instance, sutures, aneurysm coils, implants and medical instruments to promote biological responses, for instance, to stimulate growth and proliferation of cells, or healing of wounds.

The term “medical device” as used herein means a device that has one or more surfaces in contact with an organ, tissue, blood or other bodily fluid in an organism, preferably a mammal, particularly, a human. Medical devices include, for example, extracorporeal devices for use in surgery such as blood oxygenators, blood pumps, blood sensors, tubing used to carry blood, and the like which contact blood that is returned to the patient. The term can also include endoprostheses implanted in blood contact in a human or animal body, such as vascular grafts, stents, pacemaker leads, heart valves, and the like that are implanted in blood vessels or in the heart. The term can further include devices for temporary intravascular use such as catheters, guide wires, and the like that are placed in blood vessels or the heart for purposes of monitoring or repair. The term can further include nerve electrodes, muscle electrodes, implantable pulse generators, implantable drug pumps, and defibrillators. Moreover, the term medical device can include sutures, graft materials, wound coverings, nerve guides, bone wax, aneurysm coils, embolization particles, microbeads, dental implants, bone prostheses, tissue scaffolds, artificial joints or a controlled release drug delivery devices.

The surface of the medical device can be formed from any of the commonly used materials suitable for use in medical devices, such as for instance, stainless steel, titanium, platinum, tungsten, ceramics, polyurethane, polytetrafluoroethylene, extended polytetrafluoroethylene, polycarbonate, polyester, polypropylene, polyethylene, polystyrene, polyvinyl chloride, polyamide, polyacrylate, polyurethane, polyvinyl alcohol, polycaprolactone, polylactide, polyglycolide, polysiloxanes (such as 2,4,6,8-tetramethylcyclotetrasiloxane), natural rubbers, or artificial rubbers, or block polymers or copolymers thereof.

In one embodiment the invention provides a method for delivering an active peptide to a mammal, the method includes (i) providing a medical device coated on its surface with a synthetic HBGF analog, the synthetic HBGF analog being bound to the surface of the medical device by non-covalent hydrophobic bonds; and (ii) placing the medical device onto a surface of, or implanting the medical device into, the mammal.

In yet another embodiment of the above method, the method includes providing a medical device with a coating including a synthetic HBGF analog and heparin, formed by contacting the medical device with a first solution including the synthetic HBGF analog, with a second solution including heparin or an analog thereof, and with a third solution including a second synthetic HBGF, optionally wherein the second synthetic HBGF is the same as that of the first solution.

In yet another embodiment of the above method, the medical device is coated with a HBGF analog complexed to heparin.

In another embodiment of the above method, the medical device is coated with a HBGF analog with sequential complexation to heparin and a second peptide. In a preferred embodiment the second peptide is a HBGF analog.

In a further embodiment of the above method, the medical device is coated with a HBGF analog and a pre-formed heparin-peptide complex. In a preferred embodiment the peptide in the heparin-peptide complex is a HBGF analog as described above.

Heparin-Binding Growth Factors

The fibroblast growth factors, FGFs, constitute a family of related proteins controlling normal growth and differentiation of mesenchymal, epithelial, and neuroectodermal cell types. Homologs have been found in a wide variety of species. FGFs show a very high affinity to heparin and are therefore also referred to as heparin-binding growth factors (HBGFs). As used herein, the term HBGFs includes all FGFs.

Two main types of FGF are known. The first type of FGF was isolated initially from brain tissue. It was identified by its proliferation-enhancing activities for murine fibroblasts, such as 3T3 cells. Due to its basic pl the factor was named basic FGF (bFGF, or HBGF-2, heparin-binding growth factor-2) and is now generally referred to as FGF-2. This is the prototype of the FGF family.

Another type of FGF, also initially isolated from brain tissues, is acidic FGF (aFGF, also known as HBGF-1, heparin-binding growth factor-1 or HBGF-α, heparin-binding growth factor-α), now generally referred to as FGF-1. It was identified by its proliferation-enhancing activity for myoblasts.

Other fibroblast growth factors belonging to the same family include FGF-3 (or HBGF-3, heparin-binding growth factor-3, originally called int-2; see Fekete, Trends in Neurosci. 23:332 (2000)), FGF-4 (HBGF-4, heparin-binding growth factor-4, initially recognized as the product of the oncogene hst; see Sakamoto et al., Proc. Natl. Acad. Sci. USA 91:12368-72), and FGF-5 (originally called HBGF-5, see Bates et al. Biosynthesis of human fibroblast growth factor 5. Mol. Cell. Biol. 11:1840-1845 (1991); Burgess and Maciag, The heparin-binding (fibroblast) growth factor family of proteins. Ann. Rev. Biochem. 58: 575-606 (1989); and Zhan et al. The human FGF-5 oncogene encodes a novel protein related to fibroblast growth factors. Mol. Cell. Biol. 8:3487-3495 (1988)).

FGF-6 is also known as HBGF-6, and sometimes called hst-2 or oncogene hst-1 related growth factor, see lida et al. Human hst-2 (FGF-6) oncogene: cDNA cloning and characterization. Oncogene 7:303-9 (1992); and Marics et al. Characterization of the HST-related FGF-6 gene, a new member of the fibroblast growth factor gene family. Oncogene 4:335-40 (1989).

FGF-7 or K-FGF is also known as KGF or keratinocyte growth factor (See Aaronson et al. Keratinocyte growth factor is a fibroblast growth factor family member with unusual target cell specificity. Annals NY Acad. Sci. 638:62-77 (1991); Finch et al. Human KGF is FGF-related with properties of a paracrine effector of epithelial cell growth. Science 245:752-5 (1989); Marchese et al. Human keratinocyte growth factor activity on proliferation and differentiation of human keratinocytes: differentiation response distinguishes KGF from EGF family. J. Cellular Physiol. 144: 326-32 (1990)).

FGF-8 was found to be identical to androgen-induced growth factor, AIGF and has been well studied (See Blunt et al. Overlapping expression and redundant activation of mesenchymal fibroblast growth factor (FGF) receptors by alternatively spliced FGF-8 ligands. J. Biol. Chem. 272:3733-8 (1997); Dubrulle et al. FGF signaling controls somite boundary position and regulates segmentation clock control of spatiotemporal Hox gene activation. Cell 106:219-232 (2001); Gemel et al. Structure and sequence of human FGF8. Genomics 35:253-257 (1996); Tanaka et al. A novel isoform of human fibroblast growth factor 8 is induced by androgens and associated with progression of esophageal carcinoma. Dig. Dis. Sci. 46:1016-21 (2001)).

FGF-9 was originally called glia activating factor, or HBGF-9. See Miyamoto et al. Molecular cloning of a novel cytokine cDNA encoding the ninth member of the fibroblast growth factor family, which has a unique secretion pattern. Mol Cell. Biol. 13:4251-9 (1993); and Naruo et al. Novel secretory heparin-binding factors from human glioma cells (glia-activating factors) involved in glial cell growth. J. Biol Chem. 268: 2857-64 (1993).

FGF-10 is also called KGF-2, keratinocyte growth factor-2 (See Kok et al. Cloning and characterization of a cDNA encoding a novel fibroblast growth factor preferentially expressed in human heart. Biochem. Biophys. Res. Comm. 255:717-721, (1999)).

Several FGF-related factors have been described as fibroblast growth factor homologous factors (FHFs) and are also referred to as FGF-11 (FHF-3), FGF-12 (FHF-1), FGF-13 (FHF-2, see Greene et al. Identification and characterization of a novel member of the fibroblast growth factor family. Eur. J. Neurosci. 10:1911-1925 (1998)), and FGF-14 (FHF-4).

FGF-15 is expressed in the developing nervous system and was identified as a gene regulated by transcription factor E2A-Pbx1. McWhirter et al. A novel fibroblast growth factor gene expressed in the developing nervous system is a downstream target of the chimeric homeodomain oncoprotein E2A-Pbx1. Development 124:3221-3232 (1997).

FGF-16 was isolated as a cDNA clone from rat heart by homology-based polymerase chain reaction expressing an FGF of 207 amino acids. FGF-16 is 73% identical to FGF-9. Miyake et al. Structure and expression of a novel member, FGF-16, of the fibroblast growth factor family. Biochem. Biophys. Res. Commun. 243:148-152 (1998).

The cDNA encoding FGF-17 was isolated from rat embryos and encodes a protein of 216 amino acids. When expressed in 3T3 fibroblasts, mouse FGF-17 is transforming. During embryogenesis, FGF-17 is expressed at specific sites in forebrain, the midbrain-hindbrain junction, the developing skeleton and in developing arteries. See Hoshikawa et al. Structure and expression of a novel fibroblast growth factor, FGF-17, preferentially expressed in the embryonic brain. Biochem. Biophys. Res. Commun. 244:187-191 (1998); and Xu et al. Genomic structure, mapping, activity and expression of fibroblast growth factor 17. Mechanisms of Development 83:165-178 (1999).

The cDNA encoding FGF-18 was isolated from rat embryos encoding a protein of 207 amino acids. FGF-18 is a glycosylated protein and is most similar to FGF-8 and FGF-17. Injection of recombinant murine FGF-18 has been shown to induce proliferation in tissues of both epithelial and mesenchymal origin, particularly in liver and small intestine. Recombinant rat FGF-18 induces neurite outgrowth in PC12 cells. Recombinant murine FGF-18 protein stimulates proliferation in NIH 3T3 fibroblasts in vitro in a heparan sulfate-dependent manner. For general information see Hu et al. FGF-18, a novel member of the fibroblast growth factor family, stimulates hepatic and intestinal proliferation. Mol. Cell. Biol. 18:6063-6074 (1998); and Ohbayashi et al. Structure and expression of the mRNA encoding a novel fibroblast growth factor, FGF-18. J. Biol. Chem. 273:18161-18164 (1998).

FGF-19 is related distantly to other members of the FGF family. FGF-19 mRNA is expressed in several tissues including fetal cartilage, skin, and retina, as well as adult gall bladder. It is overexpressed in a colon adenocarcinoma cell line. FGF-19 is a high affinity, heparin-dependent ligand for the FGF-4 receptor. See Xie et al. FGF-19, a novel fibroblast growth factor with unique specificity for FGFR4. Cytokine 11:729-735 (1999).

FGF-20 is expressed in normal brain, particularly the cerebellum, and in some cancer cell lines. FGF-20 mRNA is expressed preferentially in the substantia nigra pars compacta. Recombinant FGF-20 protein induces DNA synthesis in a variety of cell types and is recognized by multiple FGF receptors. FGF-20 functions like an oncogene, causing a transformed phenotype when expressed in the 3T3 fibroblast cell line. These transformed cells are tumorigenic in nude mice. See Jeffers et al. Identification of a novel human fibroblast growth factor and characterization of its role in oncogenesis. Cancer Res. 61:3131-8 (2001); and Ohmachi et al. FGF-20, a novel neurotrophic factor, preferentially expressed in the substantia nigra pars compacta of rat brain. Biochem. Biophys. Res. Commun. 277:355-60 (2000).

FGF-21 was isolated from mouse embryos. FGF-21mRNA is most abundant in the liver with lower levels in the thymus. FGF-21 is most similar to human FGF-19. See Nishimura et al. Identification of a novel FGF, FGF-21, preferentially expressed in the liver. Biochim. Biophys. Acta 1492:203-6 (2000).

The cDNA encoding FGF-22 (170 amino acids) was isolated from human placenta. FGF-22 is most similar to FGF-10 and FGF-7. Murine FGF-22 mRNA is expressed preferentially in the skin. FGF-22 mRNA in the skin is found preferentially in the inner root sheath of the hair follicle. See Nakatake et al. Identification of a novel fibroblast growth factor, FGF-22, preferentially expressed in the inner root sheath of the hair follicle. Biochim. Biophys. Acta 1517:460-3 (2001).

FGF-23 is most similar to FGF-21 and FGF-19. The human FGF-23 gene maps to chromosome 12p13 linked to human FGF-6 gene. FGF-23 mRNA is expressed mainly in the brain (preferentially in the ventrolateral thalamic nucleus) and thymus at low levels. Missense mutations in the FGF-23 gene have been found in patients with autosomal dominant hypophosphataemic rickets. Overproduction of FGF23 causes tumor-induced osteomalacia, a paraneoplastic disease characterized by hypophosphatemia caused by renal phosphate wasting. See Yamashita et al. Identification of a novel fibroblast growth factor, FGF-23, preferentially expressed in the ventrolateral thalamic nucleus of the brain. Biochem. Biophys. Res. Commun. 277:494-8 (2000); and Shimada et al. Cloning and characterization of FGF23 as a causative factor of tumor-induced osteomalacia. Proc. Natl. Acad. Sci. USA 98:6500-5 (2001).

HBBM (Heparin-binding brain mitogen) was isolated initially as a heparin binding protein from brain tissues of several species and is identical to heparin-binding neurite promoting factor. See Huber et al. Amino-terminal sequences of a novel heparin-binding protein with mitogenic activity for endothelial cells from human bovine, rat, and chick brain: high interspecies homology. Neurochem. Res. 15:435-439 (1990).

HB-GAF (heparin-binding growth associated factor) is a neurotrophic and mitogenic factor identical to HBNF (heparin-binding neurite-promoting factor). See Kuo et al. Characterization of heparin-binding growth-associated factor receptor in NIH 3T3 cells. Biochem. Biophys. Res. Commun. 182:188-194 (1992).

HB-EGF (heparin-binding EGF-like factor) is found in conditioned media of cell line U937 and is also synthesized by macrophages and human vascular smooth muscle cells. HB-EGF is a monomeric heparin-binding O-glycosylated protein of 86 amino acids and is processed from a precursor of 208 amino acids. Several truncated forms of HB-EGF have been described. HB-EGF is a potent mitogen for NIH 3T3 cells, keratinocytes and smooth muscle cells, but not for endothelial cells. The mitogenic activity on smooth muscle cells is much stronger than for EGF and appears to involve interactions with cell surface heparan sulfate proteoglycans. HB-EGF is a major growth factor component of wound fluid and may play an important role in wound healing. See Abraham et al. Heparin-binding EGF-like growth factor: characterization of rat and mouse cDNA clones, protein domain conservation across species, and transcript expression in tissues. Biochem. Biophys. Res. Commun. 190:125-133 (1993); Higashiyama et al. A heparin-binding growth factor secreted by macrophage-like cells that is related to EGF. Science 251:936-9 (1991); and Marikovsky et al. Appearance of heparin-binding EGF-like growth factor in wound fluid as a response to injury. Proc. Natl. Acad. Sci. USA 90:3889-93.

HB-GAM (heparin-binding growth associated molecule) also referred to as HBNF (heparin-binding neurite promoting factor) is a protein of 15.3 kDa isolated as a heparin binding protein from brain tissues of several species. HB-GAM promotes growth of SW-13 cells in soft agar. Courty et al. Mitogenic properties of a new endothelial cell growth factor related to pleiotrophin. Biochem. Biophys. Res. Commun. 180: 145-151 (1991); and Hampton et al. Structural and functional characterization of full-length heparin-binding growth associated molecule. Mol. Biol. Cell. 3:85-93 (1992).

TGF-beta (TGF-β) exists in at least five isoforms, known TGF-β1, TGF-β2, TGF-β3, TGF-β4 and TGF-β5, all of which are not related to TGF-α. Their amino acid sequences display homologies on the order of 70-80 percent. TGF-β1 is the prevalent form and is found almost ubiquitously while the other isoforms are expressed in a more limited spectrum of cells and tissues.

TGF-beta is the prototype of a family of proteins known as the TGF-beta superfamily. This family includes inhibins, Activin A, MIS (Mullerian activating substance) and BMPs (Bone morphogenic proteins). Burt, Evolutionary grouping of the transforming growth factor-beta superfamily. Biochem. Biophys. Res. Commun. 184:590-5 (1992).

### INDUSTRIAL APPLICABILITY

The invention is further illustrated by the following non-limiting examples.

### EXAMPLE 1

The synthetic HBGF analog, F2A3, the structure of which is shown in FIG. 1, was synthesized by standard solid phase peptide synthesis methods. F2A3 has a structure according to formula II, in which the amino acid sequences of the X region, AESGDDYCVLVFTDSAWTKICDWSHFRN (SEQ ID NO:65), corresponds to the reverse sequence of the C19 peptide sequence identified by Ballinger et al. (Nature Biotechnology 17:1199 (1999)) and shown as SEQ ID NO:8. Each of the two X region peptides of SEQ ID NO:65 are covalently linked by amide bonds to a lysine residue, the lysine residues corresponding to J₁ and J₂. The J₂ Lys is bound by means of a covalent peptide bond to one terminus of a tripeptide formed from three aminohexanoic acid residues and corresponding to linker Y, providing a hydrophobic space of 18 alkyl carbon atoms. The opposite terminus of the aminohexanoic acid tripeptide is covalently bound by a peptide bond to heparin-binding peptide RKRKLERIAR (SEQ ID NO:2) corresponding to region Z.

The peptides were assembled stepwise by solid-phase synthesis on a substituted benzhydrylamine resin, using Fmoc chemistry for temporary protection of amino groups in the repetitive cycles. Branching of the chain was accomplished by stepwise growth of identical chains from the side-chain amino groups of consecutive lysyl residues. The completed peptide chains were cleaved from the resin as C-terminal amides by acidolysis, which also removed the acid-labile side-chain protecting groups.

The crude peptide preparation was first purified by heparin affinity chromatography. The crude preparation was solubilized in 10 mM HEPES (pH 7.0), loaded onto a HiTrap® Heparin HP column (Amersham Pharmacia Biotech, Piscataway, N.J., USA), and washed with 10 column volumes of 10 mM HEPES (pH 7.0). The peptide was then eluted with 2 M NaCl in 10 mM HEPES (pH 7.0), monitored by 280 nm absorbance. Peptide fractions were desalted and concentrated by loading onto Sep-Pak® C₁₈ cartridges (Waters, Milford, Mass., USA), washed with 10 column volumes of water, and then eluted with 80% acetonitrile. Eluted fractions were lyophilized, redissolved in water, and the concentration was determined by BCA® Protein Assay Kit (Pierce Endogen, Rockford, Ill., USA) using bovine serum albumin as a reference.

### EXAMPLE 2

The synthetic HBGF analog, F2A4, as shown in FIG. 2, was synthesized by standard solid phase peptide synthesis methods. The amino acid sequences of F2A4 corresponding to regions Y and Z of formula II are identical to those of F2A3 described in Example 1. The amino acid sequence RKLAVYWSSYKRSRY (SEQ ID NO:66) of the two X region peptides correspond to the reverse sequence of amino acids 115-129 of FGF-2 identified by Ray et al. (Proc. Natl. Acad. Sci. USA 94:7047-7052,1997) corresponding to SEQ ID NO:7.

The crude preparation was purified as described above in Example 1.

### EXAMPLE 3

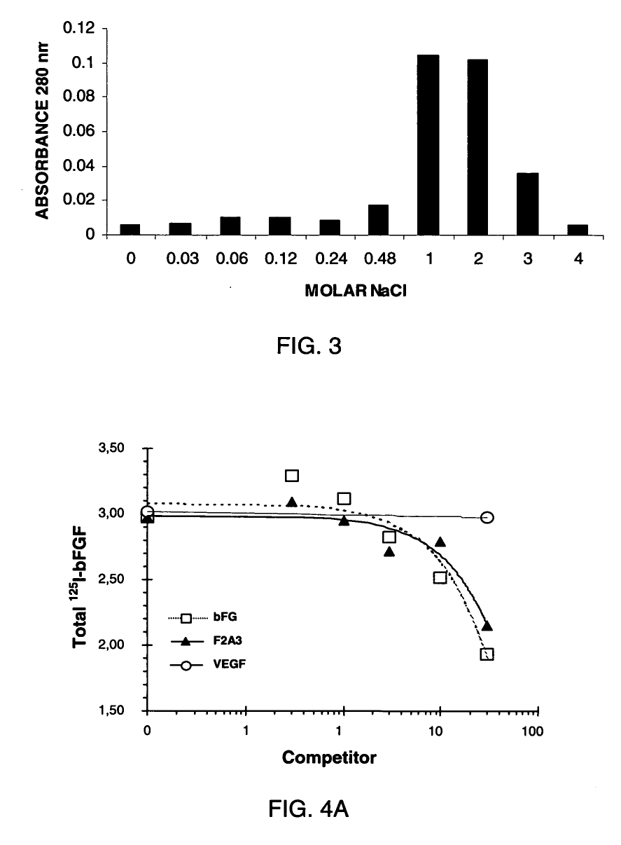

FIG. 3 shows the elution profile of F2A3 from a heparin affinity column. Mini columns were prepared with 0.5 mL heparin-agarose and washed extensively with water. F2A3 was loaded onto the column and rinsed with water. F2A3 was eluted from the column by stepwise increasing concentrations of NaCl as shown.

### EXAMPLE 4

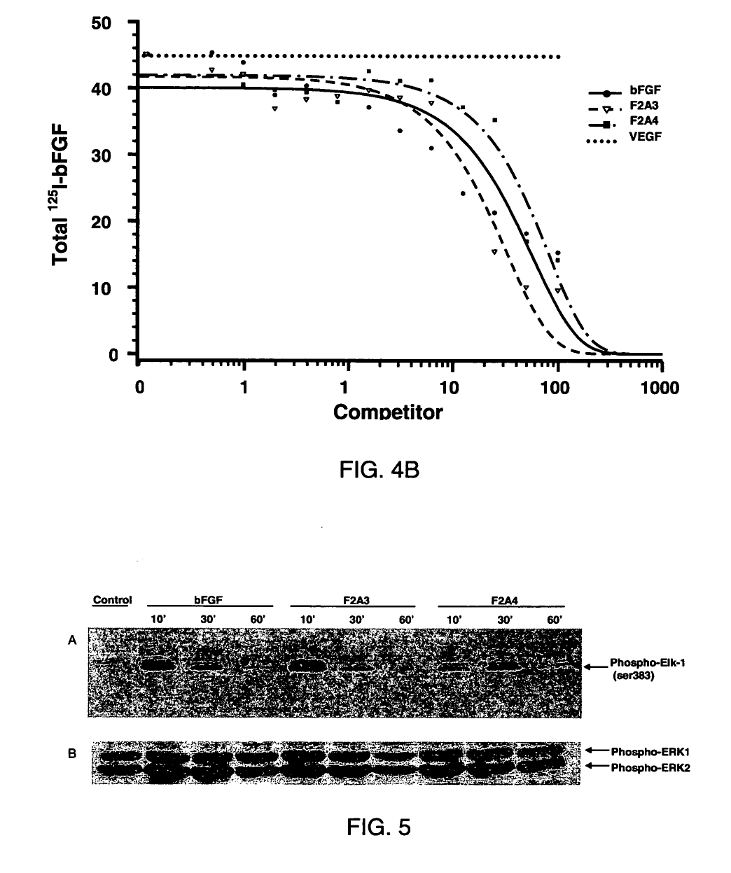

FIG. 4A shows the specific binding of F2A3 to HUVECs (Human umbilical vein endothelial cells). ¹²⁵I-bFGF was incubated with intact HUVECs in the presence of unlabeled F2A3. The bound ¹²⁵I-bFGF fraction at 4° C. was recovered from solubilized HUVEC membranes after stringent washing and quantitated in a gamma counter. F2A3 displaced ¹²⁵I-bFGF (FGF-2) bound to FGF receptors of the HUVECs, while the unrelated heparin-binding cytokine, VEGF did not. FIG. 4B shows that F2A3 and F2A4 competitively displaced ¹²⁵I-bFGF binding to a second series of cells containing FGF receptors, while the unrelated heparing-binding cytokine VEGF did not. ¹²⁵I-bFGF was incubated with cultured C3H10T1/2 fibroblasts in the presence of cold F2A3 and F2A4 for 20 minutes on ice. After stringent washing, the bound ¹²⁵I-bFGF fraction at 4° C. was recovered from solubilized cell membranes and quantitated in a gamma counter.

### EXAMPLE 5

FIG. 5 shows the equivalence of FGF-2 analogs F2A3 and F2A4 to native, recombinant FGF-2 in MAP kinase phosphorylation and activation. C3H10T1/2 cells were stimulated with 3 nM of FGF-2, F2A3 or F2A4 for 10, 30 or 60 minutes and lysed. Active MAP kinase from cell lysates were immunoprecipitated with monoclonal anti-phosphop-44/42 MAP kinase (Thr202 and Tyr204). The resulting immunoprecipitate was incubated with an Elk-1 fusion protein in the presence of ATP. Phosphorylated Elk-1 at Ser383 was visualized by western blotting using a phosphor-Elk-1 (Ser 383) antibody. To reveal the phosphorylation of MAP kinase, cell lysates were analyzed by western blotting using monoclonal anti-phosphop-44/42 MAP kinase (Thr202 and Tyr204) antibody. The results show that both F2A3 and F2A4 activate Elk-1, as does FGF-2, as shown by the phosphorylated Ser383 residue present in these samples at 10 minutes and absent from the untreated control. The level of phosphorylated Ser383 decreased successively from 10 minutes to 30 minutes and even further at 60 minutes. By contrast, the level of phosphor-ERK-1 and phosphor-ERK-2 was consistently high in the F2A3, F2A4 and FGF-2 treated samples at 10 minutes, 30 minutes and 60 minutes, whereas the control untreated sample exhibited a distinguishably lower level of each of phosphor-ERK-1 and phosphor-ERK-2. These observations show that the HBGF analogs, F2A3 and F2A4 are biologically active as FGF-2 analogs in these assays.

### EXAMPLE 6

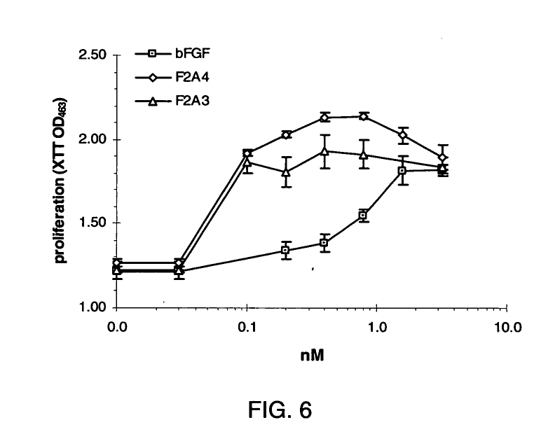

FIG. 6 shows the results of an assay for mitogenesis by F2A3 and F2A4 as compared with bFGF (FGF-2). C3H10T1/2 cells were grown in DMEM medium supplemented with 10% FBS (fetal bovine serum). Two days before the assay, cell culture medium was replaced with low serum medium (DMEM with 0.1% FBS). At the start of the assay, cells were trypsinized and a single-cell suspension was seeded onto 96-well culture plates at 1,000 cells/well. Synthetic cytokine analog peptide or recombinant human FGF-2 were added to triplicate wells (100 μL/well final volume), and culture plates were returned to a 37° C. incubator. After three days, cell proliferation was quantified by the XTT Cell Proliferation Kit II (Roche Applied Science, Indianapolis, Ind., USA) according to manufacturer's instructions.

The analogs F2A3 and F2A4 provide higher specific activities at lower concentrations than FGF-2 as shown by the results of this assay.

### EXAMPLE 7

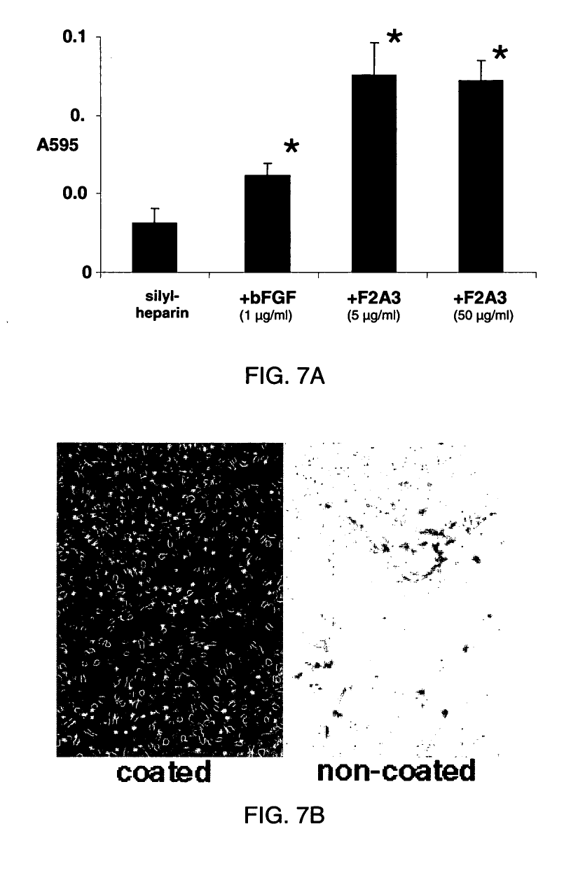

FIG. 7 shows enhancement of attachment in vitro by F2A3. Attachment of C3H10T1/2 murine fibroblasts to the wells of a polystyrene 96-well tissue culture plate coated with silyl-heparin alone or with silyl-heparin plus bFGF (FGF-2) or silyl-heparin plus F2A3 at the indicated concentrations was measured by absorbance at 595 nm after 2 hours.

Micrographs of bovine aortic endothelial cells (BEACs) grown on polycaprolactone with or without a coating of F2A3 were obtained. Cells were stained with crystal violet and photographed at 100× magnification. A substantially higher cell density of attached cells on the F2A3 coated specimen was observed.

### EXAMPLE 8

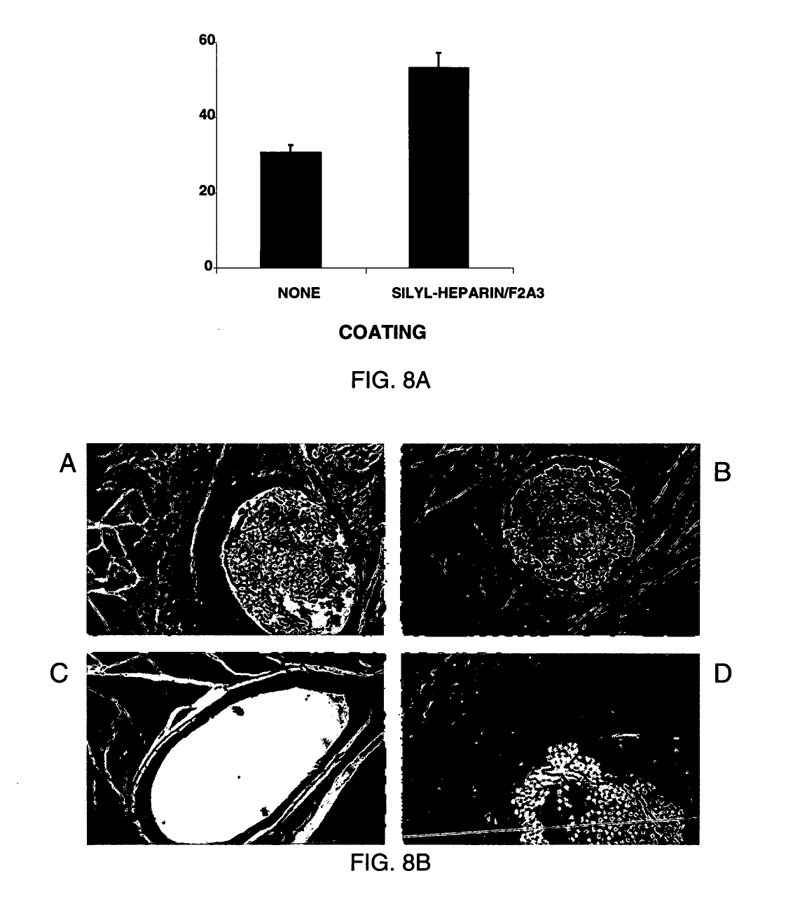

FIG. 8 shows the promotion of wound healing by locoregional delivery of F2A3 on biodegradable sutures. Bioabsorbable Vicryl® polyglycolide/lactide sutures (Ethicon Johnson & Johnson, Somerville, N.J., USA) coated to saturation with a combination silyl-heparin and F2A3 and without any coating were introduced into the thigh muscle of adult rats. After two weeks the implanted area was removed and processed for histology by routine methods. The capillaries were quantitated at a magnification of 100× and the data expressed as the average per field; as shown in FIG. 8, the Y axis depicts the number of capillaries per field. Increased granulation and angiogenesis were also observed utilizing H&E stained histological sections. Microscopic examination revealed a moderate amount of granulation after 2 weeks of rat muscle tissue where an uncoated suture was introduced. With both silyl-heparin coated sutures and F2A3 coated sutures, low to moderate granulation was found. With sutures coated with silyl-heparin and F2A3, braided PGLA fibers were evident in cross section, surrounded by a ring of granulation tissue of varying thickness, within a field of striated muscle tissue. Both silyl-heparin alone and F2A3 alone coatings reduced cellularity, compared to the control. But the combination of silyl-heparin and F2A3 caused marked fibroblast proliferation surrounding and infiltrating the braided suture, and increased endothelial cells within the granulation tissue.

### EXAMPLE 9

A synthetic HBGF analog, F1A1, was synthesized by standard solid phase peptide synthesis methods. The amino acid sequences of F1A1 corresponding to regions Y and Z of formula II are identical to those of F2A3 described in Example 1. The FGF receptor binding amino acid sequence of the two X region sequences is KKLGVFWNKEAHKKSIY (SEQ ID NO:67). This sequence is derived from a reverse sequence of amino acids bridging the beta 9 and beta 10 loop of FGF-1, which corresponds to SEQ ID NO:10.

The crude preparation was purified as described above in Example 1. The resulting analog had the following structure:

```
NH₂-K-K-Hex-Hex-Hex-R-K-R-K-L-E-R-I-A-R-amide
        Y Y
        I I
        S S
        K K
        K K
        H H
        A A
        E E
        K K
        N N
        W W
        F F
        V V
        G G
        L L
        K K
        K K
```

### EXAMPLE 10

A synthetic HBGF analog, F1A2, was synthesized by standard solid phase peptide synthesis methods. The amino acid sequences of F1A2 corresponding to regions Y and Z of formula II are identical to those of F2A3 described in Example 1. The amino acid sequence YVEGVESASLQLQIH (SEQ ID NO:68) of the two X region peptides corresponds to amino acids derived from the reverse sequence of the β-4 and β-5 region of FGF-1 (i.e., the reverse of SEQ ID NO:11). This general region is implicated in the binding of FGF-1 (Sanz, et al. Hints of nonhierarchical folding of acidic fibroblast growth factor. Biochemistry 41:1923-1933 (2002)).

The crude preparation was purified as described above in Example 1. The resulting analog had the following structure:

```
NH₂-K-K-Hex-Hex-Hex-R-K-R-K-L-E-R-I-A-R-amide
        H H
        I I
        Q Q
        L L
        Q Q
        L L
        S S
        A A
        S S
        E E
        V V
        G G
        E E
        V V
        Y Y
```

### EXAMPLE 11

A synthetic HBGF analog, F7A1, was synthesized by standard solid phase peptide synthesis methods. The amino acid sequences of F7A1 corresponding to regions Y and Z of formula II are identical to those of F2A3 described in Example 1. The amino acid sequence KQNLAVFMEGGNHTWKASAY (SEQ ID NO:69) of the two X region peptides corresponds to the reverse sequence of amino acids derived from the beta 9 and beta 10 loop of FGF-7 (i.e., the reverse of SEQ ID NO:12). Residues 91-110 which are included in this segment of FGF-7 have been described as being important for determining specificity for FGFR2llb Kim et al. (Kim et al. Colocalization of heparin and receptor binding sites on keratinocyte growth factor. Biochemistry 37:8853-8862 (1998)).

The crude preparation was purified as described above in Example 1. The resulting analog had the following structure:

```
NH₂-K-K-Hex-Hex-Hex-R-K-R-K-L-E-R-I-A-R-amide
        Y Y
        A A
        S S
        A A
        K K
        W W
        T T
        H H
        N N
        G G
        G G
        E E
        M M
        F F
        V V
        A A
        L L
        N N
        Q Q
        K K
```

### EXAMPLE 12

A synthetic HBGF analog, F7A2, was synthesized by standard solid phase peptide synthesis methods. The amino acid sequences of F7A2 corresponding to regions Y and Z of formula II are identical to those of F2A3 described in Example 1. The amino acid sequence AVIGVAVTRIEMINY (SEQ ID NO:70) of the two X region peptides corresponds to the reverse sequence of amino acids derived from the β-4 and β-5 region of FGF-7 (i.e., corresponding to SEQ ID NO:13). The loop connecting the β4-β5 strands of FGF-7 contribute to high affinity receptor binding and is critical for KGFR recognition, as determined in domain-swapping and site-directed mutagenesis experiments (Sher et al. Identification of residues important both for primary receptor binding and specificity in fibroblast growth factor-7. J. Biol. Chem. 275:34881-34886 (2000)).

The crude preparation was purified as described above in Example 1. The resulting analog had the following structure:

```
NH₂-K-K-Hex-Hex-Hex-R-K-R-K-L-E-R-I-A-R-amide
        Y Y
        N M
        I I
        M M
        E E
        I I
        R R
        T T
        V V
        A A
        V V
        G G
        I I
        V V
        A A
```

### EXAMPLE 13

A synthetic HBGF analog, F2A4-Lin, was synthesized by standard solid phase peptide synthesis methods. This analog was a linear peptide of the amino acid sequence NH₂-YRSRKYSSWYVALKRT-HexHexHex-RKRKLERIAR-amide (SEQ ID NO:71), which is a peptide of formula I, wherein n=0, T is R₁, X is YRSRKYSSWYVALKR (SEQ ID NO:7) and Z is SEQ ID NO:2, with X and Z covalently bonded by a peptide bond. The crude preparation was purified as described above in Example 1.

### EXAMPLE 14

A synthetic HBGF analog, F2A4-Sin, was synthesized by standard solid phase peptide synthesis methods. This analog is a single chain branched peptide of formula II, wherein n=0, J₁ is Lys, X is SEQ ID NO:66, Z is SEQ ID NO:2 and Y is Hex-Hex-Hex, with X covalently bonded by an amide bond to the side chain of J₁, of the following structure:

```
NH₂-K-Hex-Hex-Hex-R-K-R-K-L-G-R-I-A-R-C-amide
        Y
        R
        S
        R
        K
        Y
        S
        S
        W
        Y
        V
        A
        L
        K
        R
```

### EXAMPLE 15

Direct Coating with Synthetic HBGF Analog

Aneurysm coils were directly coated in 10 mM sodium bicarbonate containing 1 μg/mL of F2A3 of Example 1 for 1 hr at 37° C. The coils were then rinsed in water and air-dried. The coils were found to have detectable amounts of F2A3.

| TYPE OF COIL | ABSORBANCE at 450 nm |
|---|---|
| Uncoated coils (control) | 0.345 ± 0.008 |
| Coated coils | 1.791 ± 0.053 |

F2A3 on the coils was detected by an enzyme-linked immunosorbant assay (ELISA) using an avidin/biotin complex (ABC) method. In this assay the coated coils were blocked in phosphate buffered saline containing bovine serum albumin (PBS/BSA) for 30 minutes at 37° C. The coils were then immersed in PBS/BSA containing rabbit anti-F2A3 antibodies (1:500) for 1 hour at 37° C. Using a commercially available ABC kit, the coils were extensively rinsed and then incubated in PBS/BSA containing biotin conjugated goat anti-rabbit IgG (1:250) for 1 hour at 37° C. The coils were next rinsed thoroughly in water and then incubated in ABC reagents. Peroxidase was detected using a commercially available kit that contained TMB (3,3′,5,5″-tetramethylbenzidine). The absorbance of the developed chromogen was read in a microtiter plate at 450 nm.

### EXAMPLE 16

Direct Coating With Synthetic HBGF Analog and Heparin

Aneurysm coils were coated in 10 mM sodium bicarbonate containing 1 μg/mL of F2A3 of Example 1 for 1 hr at 37° C. The coils were rinsed in water and coated with 0.25% heparin in water for 30 minutes at room temperature. The coils were then rinsed in water and air-dried. The coils were found by ELISA to have detectable amounts of F2A3.

### EXAMPLE 17

Direct Coating With Synthetic HBGF Analog With Sequential Complexation to Heparin and a Secondary Peptide

Aneurysm coils were coated in 10 mM sodium bicarbonate containing 1 μg/mL of F2A3 of Example 1 for 1 hr at 37° C. The coils were rinsed in water and coated with 0.25% heparin in water for 30 minutes at room temperature. The coils were rinsed in water and immersed in phosphate buffer (pH 5.8) containing 1 μg/mL of F2A3 for 1 hour at 37° C. The coils were then rinsed in water and air-dried. The coated coils were found by ELISA to have detectable amounts of F2A3.

### EXAMPLE 18

Direct Coating With Synthetic HBGF Analog and Complexation With a Pre-Formed Heparin/Synthetic HBGF Analog Complex

Aneurysm coils were coated in 10 mM sodium bicarbonate containing 1 μg/mL of F2A3 for 1 hour at 37° C. Separately, F2A3 in a 10 mM phosphate buffer (pH 5.8) was mixed with silyl-heparin in a 4:1 or 2:1 molar ratio for 1 hour at 37° C. The coils were immersed in this solution then rinsed in water and air-dried. ELISA confirmed the presence of F2A3 on the coils.

### EXAMPLE 19

Elution of F2A3 on Coated Wafers

Stainless steel wafers were coated following the procedure outlined in examples 15, 16, and 17 above except that silyl-heparin was used instead of heparin.

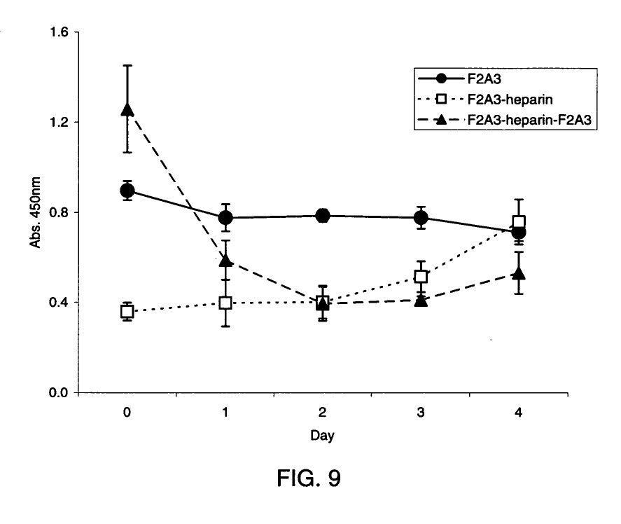

The coated wafers were placed in individual wells of 24-well plates. 0.5 mL of tissue culture medium containing 10% newborn calf serum was added to each well. Three chips from each set were removed daily for subsequent analysis. The medium was changed daily. At the end of the assay period, F2A3 was detected by ELISA, with results as shown in FIG. 9.

### EXAMPLE 20

F2A4-K-NS Coated Aneurysm Coils

The synthetic HBGF analog, F2A4-K-NS, was synthesized by standard solid phase peptide synthesis methods. The amino acid sequences of F2A4 corresponding to regions Y and Z of formula II are identical to those of F2A3 described in Example 1. The amino acid sequence YRSRKYSSWYVALKR (SEQ ID NO:7) of the two X region peptides correspond to amino acids 115-129 of FGF-2 identified by Ray et al. (Proc. Natl. Acad. Sci. USA 94:7047-7052, 1997). The construct was a branched peptide of the structure YRSRKYSSWYVALKRK(H-YRSRKYSSWYVALKR)-Ahx-Ahx-Ahx-RKRKLERIAR-NH₂, and was synthesized by standard solid phase peptide synthesis methods. In the compound YRSRKYSSWYVALKRK(H-YRSRKYSSWYVALKR)-Ahx-Ahx-Ahx-RKRKLERIAR-N H₂, the R₁ group of formula I was a single trifunctional amino acid residue, here a diamine amino acid, lysine (K). The peptide of Example 20 has an estimated molecular weight of 5681.

The peptide of Example 20 was assembled stepwise by solid-phase synthesis on a substituted resin, using Fmoc chemistry for temporary protection of amino groups in the repetitive cycles. Protecting groups were used as required. Branching of the chain was accomplished by stepwise growth of identical chains from the alpha amino group and side-chain amino group of a single lysyl residue. The completed peptide chain was cleaved from the resin as C-terminal amides by acidolysis, which also removed the acid-labile side-chain protecting groups. The peptide of Example 20 was purified by reverse phase HPLC using a C₁₈ column in a continuous gradient elution of 0-60% B over 60 minutes, run at 1 mL/min, where A was 0.1% trifluoroacetate in water and B was 0.1% trifluoroacetate in acetonitrile. The general structure of the compound of Example 20 is shown below:

```
NH₂-Y-R-S-R-K-Y-S-S-W-Y-V-A-L-K-R-K-Hex-Hex-Hex-R-K-R-K-L-E-R-I-A-R-amide
                                                                  R
                                                                  K
                                                                  L
                                                                  A
                                                                  V
                                                                  Y
                                                                  W
                                                                  S
                                                                  S
                                                                  Y
                                                                  K
                                                                  R
                                                                  S
                                                                  R
                                                                  Y
```

The peptide F2A4-K-NS was used to coat aneurysm coils. All procedures were performed aseptically, with coils handled using sterile forceps. All procedures were performed in a hood, with all solutions either sterile filtered or autoclaved. Incubations were performed in tubes with the cap in place. An aneurysm coil was placed in a sterile Eppendorf tube. A solution was added containing 10 mM mM NaHCO₃ at approximately pH 8.0, with F2A4 at a concentration of 10 μg/coil. The coil was allowed to incubate in the solution for one hour at 37° C., the liquid removed, and the coil dried at 37° C.

A solution was then added containing 0.01% sodium heparin in water, with the coil was allowed to incubate in the solution for ten minutes at 37° C., the liquid removed, and the coil dried at 37° C.

Optionally a final solution containing F2A4 was added, at the same concentration as the first solution, and allowed coil was allowed to incubate in the solution for thirty minutes at 37° C., the liquid removed, and the coil dried at 37° C. The coil was then sterile packaged for use.

The preceding examples can be repeated with similar success by substituting the generically or specifically described reactants and/or operating conditions of this invention for those used in the preceding examples.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above are hereby incorporated by reference.

We claim:

1. A method of coating a medical device on a surface thereof, the method comprising the steps of:
contacting a medical device with a first solution comprising a synthetic heparin-binding growth factor analog comprising at least one sequence of amino acid residues binding a heparin-binding growth factor receptor (HBGFR), a hydrophobic linker region and a heparin-binding region; and
contacting the medical device with a second solution comprising a heparin-containing compound.

2. The method of claim 1, further comprising the step of contacting the medical device with a third solution comprising a heparin-binding growth factor analog comprising at least one sequence of amino acid residues binding a HBGFR, a hydrophobic linker region and a heparin-binding region.

3. The method of claim 2, wherein the heparin-binding growth factor analog of the first solution is the same as the heparin-binding growth factor analog of the third solution.

4. The method of claim 2, wherein the steps are ordered such that the medical device is first contacted with the first solution, second contacted with the second solution, and third contacted with the third solution.

5. The method of claim 1 wherein the medical device is a suture, graft material, wound covering, nerve guide, bone wax, aneurysm coil, embolization particle, microbead, stent, dental implant, bone prosthesis, tissue scaffold or controlled release drug delivery device.

6. The method of claim 5 wherein the surface of the medical device is stainless steel, titanium, platinum, tungsten, ceramics, polyurethane, polytetrafluoroethylene, extended polytetrafluoroethylene, polycarbonate, polyester, polypropylene, polyethylene, polystyrene, polyvinyl chloride, polyamide, polyacrylate, polyurethane, polyvinyl alcohol, polycaprolactone, polyactide, polyglycolide, polysiloxane, natural rubber, artificial rubber, a block polymer, or a copolymer of block polymers.

7. The method of claim 1 wherein the heparin-containing compound comprises a silyl-heparin compound.

8. The method of claim 1 wherein the at least one sequence of amino acid residues binding a HBGFR is a sequence selected from the group consisting of SEQ ID NO:7 to SEQ ID NO:70.

9. The method of claim 1 wherein the heparin-binding region is a sequence of amino acid residues selected from the group consisting of SEQ ID NO:1 to SEQ ID NO:6.

10. The method of claim 1 wherein the synthetic heparin-binding growth factor analog comprising at least one sequence of amino acid residues binding a HBGFR, a hydrophobic linker region and a heparin-binding region is a construct of any of formulas I to XI.

11. A method of coating a medical device on a surface thereof, the method comprising the step of:
contacting a medical device with a first solution comprising a heparin-containing compound and a synthetic heparin-binding growth factor analog comprising at least one sequence of amino acid residues binding a heparin-binding growth factor receptor (HBGFR), a hydrophobic linker region and a heparin-binding region.

12. The method of claim 11, further comprising the step of contacting the medical device with a second solution comprising a heparin-binding growth factor analog comprising at least one sequence of amino acid residues binding a heparin-binding growth factor receptor (HBGFR), a hydrophobic linker region and a heparin-binding region.

13. A coated medical device comprising a medical device member and a coating comprising the product of application of a synthetic heparin-binding growth factor analog comprising at least one sequence of amino acid residues binding a HBGFR, a hydrophobic linker region and a heparin-binding region and sequential application of a heparin-containing compound.

14. The coated medical device of claim 13 wherein the coating further comprises the product of a sequential application of a synthetic heparin-binding growth factor analog comprising at least one sequence of amino acid residues binding a HBGFR, a hydrophobic linker region and a heparin-binding region.

15. The coated medical device of claim 13 wherein the at least one sequence of amino acid residues binding a HBGFR is a sequence selected from the group consisting of SEQ ID NO:7 to SEQ ID NO:70.

16. The coated medical device of claim 13 wherein the heparin-binding region is a sequence of amino acid residues selected from the group consisting of SEQ ID NO:1 to SEQ ID NO:6.

17. The coated medical device of claim 13 wherein the hydrophobic linker region comprises amino hexanoic acid.

18. The coated medical device of claim 13 wherein the heparin-containing compound comprises a silyl-heparin compound.

19. The coated medical device of claim 18 wherein the silyl-heparin compound is benzyl-bis(dimethylsilylmethyl)oxycarbamoyl-heparin.

20. The coated medical device of claim 13 wherein the coating binds a cell surface receptor.

21. The coated medical device of claim 13 wherein the coating supports cell attachment.

22. The coated medical device of claim 13 wherein the coating is vaso-occlusive.

23. The coated medical device of claim 13 wherein the medical device is a suture, graft material, wound covering, nerve guide, bone wax, aneurysm coil, embolization particle, microbead, stent, dental implant, bone prosthesis, tissue scaffold or controlled release drug delivery device.

24. A coating for a medical device, comprising a composition formed in situ from the sequential application to the medical device of a first solution comprising a synthetic heparin-binding growth factor analog comprising at least one sequence of amino acid residues binding a HBGFR, a hydrophobic linker region and a heparin-binding region and a second solution comprising a heparin-containing compound.

25. The coating of claim 24, further comprising a composition formed in situ from the sequential application to the medical device of a third solution comprising a heparin-binding growth factor analog comprising at least one sequence of amino acid residues binding a HBGFR, a hydrophobic linker region and a heparin-binding region.


SEQUENCE LISTING (71 sequences)

SEQ ID NO: 1 - protein, 6 aa, Artificial
  artificial heparin-binding sequence
XXXXXX

SEQ ID NO: 2 - protein, 10 aa, Artificial
  synthetic heparin-binding sequence
RKRKLERIAR

SEQ ID NO: 3 - protein, 10 aa, Artificial
  synthetic heparin-binding sequence
RKRKLGRIAR

SEQ ID NO: 4 - protein, 10 aa, Artificial
  synthetic heparin-binding sequence
RKRKLWRARA

SEQ ID NO: 5 - protein, 9 aa, Artificial
  synthetic heparin-binding sequence
RKRLDRIAR

SEQ ID NO: 6 - protein, 11 aa, Artificial
  synthetic heparin-binding sequence
RKRKLERIARC

SEQ ID NO: 7 - protein, 15 aa, Artificial
  synthetic FGF-2 analog
YRSRKYTSWYVALKR

SEQ ID NO: 8 - protein, 28 aa, Artificial
  synthetic FGF-2 analog
NRFHSWDCIKTWASDTFVLVCYDDGSEA

SEQ ID NO: 9 - protein, 15 aa, Artificial
  synthetic FGF-2 analog
HIKLQLQAEERGVVS

SEQ ID NO: 10 - protein, 17 aa, Artificial
  synthetic FGF-1 analog
YISKKHAEKNWFVGLKK

SEQ ID NO: 11 - protein, 15 aa, Artificial
  synthetic FGF-1 analog
HIQLQLSAESVGEVY

SEQ ID NO: 12 - protein, 20 aa, Artificial
  synthetic FGF-7 analog
YASAKWTHNGGEMFVALNQK

SEQ ID NO: 13 - protein, 15 aa, Artificial
  synthetic FGF-7 analog
YNIMEIRTVAVGIVA

SEQ ID NO: 14 - protein, 20 aa, Artificial
  synthetic FGF-10 analog
YASFNWQHNGRQMYVALNQK

SEQ ID NO: 15 - protein, 19 aa, Artificial
  synthetic FGF-22 analog
YASQRWRRRGQPNLALDRR

SEQ ID NO: 16 - protein, 21 aa, Artificial
  synthetic FGF-9 analog
YSSNLYKHVDTGRRYYVALNK

SEQ ID NO: 17 - protein, 20 aa, Artificial
  synthetic FGF-16 analog
YASTLYKHSDSERQYVALNK

SEQ ID NO: 18 - protein, 20 aa, Artificial
  synthetic FGF-20 analog
YSSNIYKHGDTGRRFVALNK

SEQ ID NO: 19 - protein, 16 aa, Artificial
  synthetic FGF-4 analog
YESYKYPGMFIALSKN

SEQ ID NO: 20 - protein, 17 aa, Artificial
  synthetic FGF-6 analog
YESDLYQGTYILSKYGR

SEQ ID NO: 21 - protein, 20 aa, Artificial
  synthetic FGF-12 analog
YSSTLYRQQESGRAWFLGNK

SEQ ID NO: 22 - protein, 21 aa, Artificial
  synthetic FGF-14 analog
YSSMLYRQQESGRAWFLGLNK

SEQ ID NO: 23 - protein, 21 aa, Artificial
  synthetic FGF-13 analog
YSSMIYRQQQSGRGWYLGLNK

SEQ ID NO: 24 - protein, 19 aa, Artificial
  synthetic FGF-11 analog
YASALYRQRRSGRAWYLDK

SEQ ID NO: 25 - protein, 20 aa, Artificial
  synthetic VEGF analog
APMAEGGGQNHHEVVKFMDV

SEQ ID NO: 26 - protein, 11 aa, Artificial
  synthetic VEGF analog
GATWLPPNPTK

SEQ ID NO: 27 - protein, 20 aa, Artificial
  synthetic VEGF analog
NFLLSWVHWSLALLLYLHHA

SEQ ID NO: 28 - protein, 13 aa, Artificial
  synthetic BMP analog
LYVDFSDVGWNDW

SEQ ID NO: 29 - protein, 15 aa, Artificial
  synthetic BMP analog
AISMLYLDENEKVVL

SEQ ID NO: 30 - protein, 17 aa, Artificial
  synthetic BMP analog
ISMLYLDENEKVVLKNY

SEQ ID NO: 31 - protein, 15 aa, Artificial
  synthetic BMP analog
EKVVLKNYQDMVVEG

SEQ ID NO: 32 - protein, 16 aa, Artificial
  synthetic BMP analog
LVVKENEDLYLMSIAC

SEQ ID NO: 33 - protein, 16 aa, Artificial
  synthetic BMP analog
AFYCHGECPFPLADHL

SEQ ID NO: 34 - protein, 23 aa, Artificial
  synthetic BMP analog
PFPLADHLNSTNHAIVQTLVNSV

SEQ ID NO: 35 - protein, 21 aa, Artificial
  synthetic TGF-beta1 analog
IVYYVGRKPKVEQLSNMIVRS

SEQ ID NO: 36 - protein, 22 aa, Artificial
  synthetic TGF-beta2 analog
TILYYIGKTPKIEQLSNMIVKS

SEQ ID NO: 37 - protein, 21 aa, Artificial
  synthetic TGF-beta3 analog
LTILYYVGRTPKVEQLSNMVV

SEQ ID NO: 38 - protein, 23 aa, Artificial
  synthetic BMP-2 analog
AISMLYLDENEKVVLKNYQDMVV

SEQ ID NO: 39 - protein, 24 aa, Artificial
  synthetic BMP-3 analog
SSLSILFFDENKNVVLKVYPNMTV

SEQ ID NO: 40 - protein, 24 aa, Artificial
  synthetic BMP-3beta analog
NSLGVLFLDENRNVVLKVYPNMSV

SEQ ID NO: 41 - protein, 23 aa, Artificial
  synthetic BMP-4 analog
AISMLYLDEYDKVVLKNYQEMVV

SEQ ID NO: 42 - protein, 23 aa, Artificial
  synthetic BMP-5 analog
AISVLYFDDSSNVILKKYRNMVV

SEQ ID NO: 43 - protein, 23 aa, Artificial
  synthetic BMP-6 analog
AISVLYFDDNSNVILKKYRNMVV

SEQ ID NO: 44 - protein, 23 aa, Artificial
  synthetic BMP-7 analog
AISVLYFDDSSNVILKKYRNMVV

SEQ ID NO: 45 - protein, 23 aa, Artificial
  synthetic BMP-8 analog
ATSVLYYDSSNNVILRKARNMVV

SEQ ID NO: 46 - protein, 23 aa, Artificial
  synthetic BMP-9 analog
ISVLYKDDMGVPTLKYHYEGMSV

SEQ ID NO: 47 - protein, 22 aa, Artificial
  synthetic BMP-10 analog
ISILYLDKGVVTYKFKYEGMAV

SEQ ID NO: 48 - protein, 22 aa, Artificial
  synthetic BMP-11 analog
INMLYFNDKQQIIYGKIPGMVV

SEQ ID NO: 49 - protein, 22 aa, Artificial
  synthetic BMP-12 analog
ISILYIDAANNVVYKQYEDMVV

SEQ ID NO: 50 - protein, 22 aa, Artificial
  synthetic BMP-13 analog
ISILYIDAGNNVVYKQYEDMVV

SEQ ID NO: 51 - protein, 22 aa, Artificial
  synthetic BMP-14 analog
ISILFIDSANNVVYKQYEDMVV

SEQ ID NO: 52 - protein, 22 aa, Artificial
  synthetic BMP-15 analog
ISVLMIEANGSILYKEYEGMIA

SEQ ID NO: 53 - protein, 22 aa, Artificial
  synthetic GDF-1 analog
ISVLFFDNSDNVVLRQYEDMVV

SEQ ID NO: 54 - protein, 22 aa, Artificial
  synthetic GDF-3 analog
ISMLYQDNNDNVILRHYEDMVV

SEQ ID NO: 55 - protein, 22 aa, Artificial
  synthetic GDF-8 analog
INMYLFNGKEQIIYGKIPAMVV

SEQ ID NO: 56 - protein, 22 aa, Artificial
  synthetic GDF-9 analog
LSVLTIEPDGSIAYKEYEDMIA

SEQ ID NO: 57 - protein, 15 aa, Artificial
  synthetic reverse BMP-2 analog
LVVKENEDLYLMSIA

SEQ ID NO: 58 - protein, 17 aa, Artificial
  synthetic reverse BMP-2 analog
YNKLVVKENEDLYLMSI

SEQ ID NO: 59 - protein, 13 aa, Artificial
  synthetic reverse BMP-7 analog
KKLIVNSSEDFYL

SEQ ID NO: 60 - protein, 13 aa, Artificial
  synthetic reverse BMP-2 analog
WDNWGVDSFDVYL

SEQ ID NO: 61 - protein, 15 aa, Artificial
  synthetic reverse BMP-2 analog
GEVVMDQYNKLVVKE

SEQ ID NO: 62 - protein, 16 aa, Artificial
  synthetic reverse BMP-2 analog
LHDALPFPCEGHCYFA

SEQ ID NO: 63 - protein, 23 aa, Artificial
  synthetic reverse BMP-2 analog
VSNVLTQVIAHNTSNLHDALPFP

SEQ ID NO: 64 - protein, 16 aa, Artificial
  synthetic reverse BMP-2 analog
LVVKENEDLYLMSIAC

SEQ ID NO: 65 - protein, 28 aa, Artificial
  Synthetic reverse FGF analog
AESGDDYCVLVFTDSAWTKICDWSHFRN

SEQ ID NO: 66 - protein, 15 aa, Artificial
  synthetic reverse FGF analog
RKLAVYWSSYKRSRY

SEQ ID NO: 67 - protein, 17 aa, Artificial
  Synthetic reverse FGF analog
KKLGVFWNKEAHKKSIY

SEQ ID NO: 68 - protein, 15 aa, Artificial
  Synthetic reverse FGF analog
YVEGVESASLQLQIH

SEQ ID NO: 69 - protein, 20 aa, Artificial
  Synthetic reverse FGF analog
KQNLAVFMEGGNHTWKASAY

SEQ ID NO: 70 - protein, 15 aa, Artificial
  Synthetic reverse FGF analog
AVIGVAVTRIEMINY

SEQ ID NO: 71 - protein, 29 aa, Artificial
  Synthetic HBGF analog with FGF sequence, hydrophobic linker region and heparin-binding region
YRSRKYSSWYVALKRTXXXRKRKLERIAR